(19) United States
(12) Patent Application Publication
(10) Pub. No.: US 2025/0292869 A1
(43) Pub. Date: Sep. 18, 2025

(54) UTILIZING THE THERMODYNAMICS OF DNA METHYLATION PROCESSES

(71) Applicant: THE PENN STATE RESEARCH FOUNDATION, University Park, PA (US)

(72) Inventors: Sally Mackenzie, University Park, PA (US); Robersy Sanchez Rodriguez, University Park, PA (US)

(21) Appl. No.: 19/182,180

(22) Filed: Apr. 17, 2025

Related U.S. Application Data
(63) Continuation of application No. PCT/US2023/077135, filed on Oct. 18, 2023.
(60) Provisional application No. 63/380,180, filed on Oct. 19, 2022.

(51) Int. Cl. G16B 30/00; G06N 20/10; G16B 5/20; G16B 20/50; G16B 40/20

(52) U.S. Cl. CPC G16B 30/00; G06N 20/10; G16B 5/20; G16B 20/50; G16B 40/20

(57) ABSTRACT

A framework consistent with thermodynamic principles to decipher the DNA methylation process utilizes a probability density function of DNA methylation information-divergence, summarizes the statistical biophysics underlying spontaneous methylation background, and bears on the channel capacity of molecular machines conforming to Shannon's capacity theorem. Contributions from the molecular machine (enzyme) logical operations to Gibbs entropy (S) and Helmholtz free energy (F) are intrinsic. Biomedical and biopharmaceutical industrial applications are achievable by way of estimating S on methylome datasets. As a thermodynamic state variable, the individual methylome entropy is completely determined by the current state of the system, which in biological terms translates to a correspondence between estimated entropy values and observable phenotypic state. Analysis of entropy fluctuations on experimental datasets revealed the existence of restrictions on the magnitude of genome-wide methylation changes during organismal response to environmental changes, thereby allowing for earlier-stage diagnostics and prediction of epigenetic state changes.

### CROSS REFERENCE TO RELATED APPLICATIONS

This application claims priority to PCT Patent App. No. PCT/US2023/077135, filed Oct. 18, 2023; and U.S. provisional patent application Ser. No. 63/380,180, filed Oct. 19, 2022. These applications are hereby incorporated by reference in their entireties herein, including without limitation, the specification, claims, and abstract, as well as any figures, tables, appendices, or drawings thereof.

### STATEMENT REGARDING FEDERALLY SPONSORED RESEARCH OR DEVELOPMENT

This invention was made with government support under Grant No. GM134056 awarded by the National Institutes of Health. The Government has certain rights in the invention.

### TECHNICAL FIELD

The present disclosure relates to, but is not limited to relating to, DNA methylation, information thermodynamics, and epigenetics. More particularly, but not exclusively, the present disclosure recognizes that the utilization of the thermodynamics of DNA methylation processes has industrial applications.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Some of the subject matter of this disclosure relates to some of the subject matter of PCT/US2023/064913, filed on Mar. 24, 2023 under the names of the same Applicant and same inventors. PCT/US2023/064913 claimed priority to U.S. provisional patent application Ser. No. 63/323,690, filed Mar. 25, 2022. U.S. provisional patent application Ser. No. 63/323,690 is hereby incorporated by reference in its entirety herein, including without limitation, the specification, claims, and abstract, as well as any figures, tables, appendices, or drawings thereof.

DNA methylation is an epigenetic mechanism that plays important roles in various biological processes including transcriptional and post-transcriptional regulation, genomic imprinting, aging, and stress response to environmental changes and disease.

Cytosine DNA methylation is one of the most well-characterized epigenetic modifications to date. It plays important roles in various biological processes, including X-chromosome inactivation, genomic imprinting, transposon suppression, transcriptional regulation, and the aging process. Additionally, DNA methylation plays an important role in preserving DNA stability, which implies that the most frequent methylation changes serve to preserve thermodynamic stability of DNA molecules.

The present inventors have previously explained in detail how these methylation changes comprise the background activity that must be distinguished from targeted differentially methylated positions (DMPs) that are introduced by the methylation regulatory machinery. See Sanchez et al., “Discrimination of DNA Methylation Signal from Background Variation for Clinical Diagnostics”, Int. J. Mol. Sci. 20, 5343 (2019), which is hereby incorporated by reference in its entirety herein.

When evaluating samples from a single species under various experimental conditions, it is not difficult, through data analysis and simulation, to find evidence of differential methylation activity in control populations. See id. These DMPs are presumed to derive from fluctuations inherent to any stochastic process, a property summarized by the fluctuation theorem. Regardless of constant environment, statistically significant methylation changes are found in a control population with probability greater than zero, implying that stochasticity of the methylation process derives from the inherent stochasticity of biochemical systems. Spontaneous natural methylation variation (“noise”) is expected within multicellular organisms, while methylation regulatory machinery (“signal”) directs organismal adaption to micro- and macro-environmental fluctuation and during development.

The present inventors have developed models for the probability distribution of methylation variation (noise plus signal), expressed as information divergences of methylation levels, were derived for a constrained scenario on a statistical physical basis. See Sanchez et al., “Information thermodynamics of cytosine DNA methylation.” PLOS One 11, c0150427 (2016), which is hereby incorporated by reference in its entirety herein. Background methylation variation could be described in terms of a generalized gamma probability distribution or a member of a generalized gamma distribution family. However, such modeling, see id., only works as a transfer function where model parameters remain undefined, which is useful for practical applications in modeling the system's output for each possible input, but not for understanding the thermodynamics of the methylation process.

A formal derivation of the generalized gamma distribution model for the cytosine DNA methylation process considers the continuous action of the Second Law of thermodynamics on biological processes and the consequent application of Jaynes' Maximum Entropy Principle, following from Jaynes' attempt to characterize an information-theoretical account of the Second Law. Statistical physical assumptions are set on the channel capacity of molecular machines (typically enzymes or macromolecular structures integrated by DNA and enzymes), which is closely related to Shannon's channel capacity. Biological molecular machines are assumed with energy scales comparable to the thermal energy at ambient temperature with sensitivity to thermal fluctuation. This modeling can provide a physical interpretation for parameters not previously undertaken.

Thus, there exists a need in the art for practical applications, that on thermodynamic and informational bases, that utilize the probability distributions of the information divergences of methylation levels (members of the generalized gamma probability distribution family) that accurately describe the methylation process. For example, individual methylation system entropy and Helmholtz free energy can help assess the biological implications of the theory for analysis of methylation variations in plants and animals.

### SUMMARY

DNA methylation plays a fundamental epigenetic role in the development and environmental responsiveness of multicellular organisms. Proper assessment of the thermodynamics of methylation variation in natural populations is beneficial to understanding system dynamics and discriminating methylation regulatory signal from background “noise”. Modeling founded on well-established physical principles can be an indispensable step for systematizing scientific approaches and improving scientific insight and model prediction accuracy, depending on the application. Resolving the thermodynamics of DNA methylation in cell populations impacts the accuracy and confidence of model predictions, particularly for clinical diagnostics and prognosis.

The present disclosure shows the application of maximum entropy principle and constraints derived from the molecular machine channel capacity describe the methylation process not only in terms of a probability distribution ƒ(E) of energy dissipated E but also as the probability that the integrity of the DNA methylation message is preserved under environmental fluctuation (e.g., diseases, a drug treatment, lifestyle, climate changes, etc.). Formally, following Shannon's theory, the analytically derived probability distribution ƒ(E) can be re-interpreted as the probability P_{y}(χ) such that, if the recovered message at the receiving point is y, the information divergence between y and the original message x produced by the source is χ.

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

While experimental sciences can only provide evidence supporting research hypotheses and demonstrations are only possible in a mathematical theoretical framework, it is a further object, feature, and/or advantage of the present disclosure to provide support for the epigenetic reprogramming (i) induced by msh1 mutation in the plant Arabidopsis thaliana and (ii) induced by different cancer types in humans, respectively. Nonconformity to the rules set forth in the present disclosure occurred only in dysfunctional states represented by Arabidopsis methyltransferase mutant met1 and human cancer cells was. In patients with different types of cancer, results suggest that a significant information loss occurs in the transition from differentiated (healthy) tissues to cancer cells.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIGS. 1A-1B shows a graphical summary of information thermodynamics of the methylation process and its application to methylation analysis.

FIG. 1A is a flow chart in compliance with thermodynamic entropy. The flow chart shows the application of Jaynes' Maximum Entropy Principle (MEP) leads to Boltzmann distribution as most probable for the methylation system. Criteria derived from molecular machine channel capacity and further maximum likelihood estimations lead to the theoretical derivation of a generalized gamma distribution model as best to describe the genome-wide methylation changes observable in an individual dataset, expressed in terms of the information divergence of methylation changes: χ:E=χk_{B}Tθ⁻¹. The state of the methylation system is described by generalized gamma probability density function, from which an analytical expression for entropy of the methylation system is derived.

FIG. 1B shows a diagram representation of methylome variation based on empirical data analysis. Analysis of experimental datasets supports the best fitted model to be a generalized gamma distribution model or a member of the generalized gamma probability distribution family. Molecular thermal noise generated by biochemical processes induces methylation changes to stabilize the DNA molecule, and this background variation conforms to laws of statistical physics (left side of FIG. 1B). The methylation regulatory signal is a response to developmental and/or environmental conditions and can be discerned as starting from the receiver's threshold (here denoted χ_{α=0.05}) and determined by each individual probability density function. With probability greater than zero, highlighted in the colored area below the right portion of the curve, there is a risk of false positive classification for a subset of cytosine sites, even when absent from the data sample, because methylation fluctuations are also present in the background variation of control samples. Signal detection and machine-learning are implemented to reduce the risk of false positives by estimation of an optimal cutoff value χ_{cutoff}≥χ_{α=0.05} to discriminate methylation signal induced by treatment or disease from naturally occurring background variation, permitting the classification of legitimate differentially methylated positions (DMPs). Thus, the probability density function of χ is the null hypothesis applied to identify DMPs. As suggested by the graphic, DMPs provide only a small informational contribution to the system entropy, which is a thermodynamic state variable of the system.

FIGS. 2A-2F shows an evaluation of entropy fluctuations in experimental datasets.

FIG. 2A shows a regression analysis −k_{B}⁻¹|S| versus the expected value (mean) v=<χ> of the J-information-divergence χ for datasets from Arabidopsis memory lines over six generations and the met1 mutant (in the subplot).

FIG. 2B shows regression analysis −k_{B}⁻¹|S| versus v on human datasets from patient with different types of cancer and tissue controls. Regression analysis in both panels support the linear model −k_{B}⁻¹|S|=ρ₁v+ρ₀. Only dysfunctional situations, such as Arabidopsis met1 mutant, human breast cancer, human metastasis (in red), or undifferentiated stem cells (hesc, in magenta), fail to conform to −k_{B}⁻¹|S|=ρ₁v+ρ₀. The statistically significant regression analysis −k_{B}⁻¹|S| versus v leads to consider the regression e^{-k}^{_{B}}^{-1|S|} versus e^{-v}.

FIG. 2C shows regression analysis e^{-k}^{_{B}}^{-1|S|} versus e^{-v} for datasets from Arabidopsis memory lines over six generations and the met1 mutant (in the subplot).

FIG. 2D shows regression analysis e^{-k}^{_{B}}^{-1|S|} versus e^{-v} for datasets on human datasets from patient with different types of cancer and tissue controls. Regression analysis in both FIGS. 2C and 2D support, up to the experimental error, the regression model e^{-k}^{_{B}}^{-1|S|}=−ηe^{-v}+η or, equivalently, e^{-k}^{_{B}}^{-1|S|}=η(1−e^{-v}). Only dysfunctional situations, such as Arabidopsis met1 mutant, human breast cancer, human metastasis (in red), or undifferentiated stem cells (hesc, in magenta), fail to conform to e^{-k}^{_{B}}^{-1|S|}=η(1−e^{-v}).

If the regression model e^{-k}^{_{B}}^{-1|S|}=η(1−e^{-v}) is valid, then after introducing the approach e^{-v}≈1−v in the model, the new model derived e^{-k}^{_{B}}^{-1|S|}=ηv must be valid up to the experimental error as well.

FIG. 2E shows regression analysis e^{-k}^{_{B}}^{-1|S|} versus v for datasets from Arabidopsis memory lines over six generations and the met1 mutant (in the subplot).

FIG. 2F shows regression analysis e^{-k}^{_{B}}^{-1|S|} versus v on human datasets from patient with different types of cancer and tissue controls. Regression analysis in both panels support e^{-k}^{_{B}}^{-1|S|}=ηv. Only dysfunctional situations, such as Arabidopsis met1 mutant, human breast cancer, human metastasis (in red), or undifferentiated stem cells (hesc, in magenta), fail to conform to e^{-k}^{_{B}}^{-1|S|}=ηv.

FIG. 3A shows a boxplot with sum of Boltzmann's factors

$${e}^{- \frac{\vert S \vert}{{k}_{B}}} + {e}^{- v}$$

in human datasets. The graphic shows that breast cancer and metastasis, as well as stem cells, fail to conform to [symbol] e^{-k}^{_{B}}^{-1|S|}+e^{-v} [symbol] =λ.

FIG. 3B shows a bar plot with estimations of the average of Boltzmann's factors sum

$${e}^{- \frac{\vert S \vert}{{k}_{B}}} + {e}^{- v}$$

for entire sets of Arabidopsis and human samples. The number of individuals for each chromosome are given on each bar in white. The statistical summaries for the five Arabidopsis chromosomes and ten human somatic chromosomes are shown at top. The error bars correspond to standard deviation estimates on each chromosome. Results indicate statistically nonsignificant differences for the Boltzmann's factors sums estimated for Arabidopsis and human datasets, supporting [symbol] e^{-k}^{_{B}}^{-1|S|}+e^{-v} [symbol] =λ.

FIGS. 4A-4B show chromosome Gibbs entropy estimated on the groups: TD and ASD. FIG. 4A relates to males. FIG. 4B relates to females. The best probabilistic generalized gamma model for each chromosome was used for the estimation of Gibbs entropy according to

$$S = {k}_{B} ( ln \frac{β Γ ( δ )}{α} + ψ ( δ ) ( \frac{1}{α} - δ ) + δ ) .$$

The units of the entropy values in the graphics are: Joule×Kelvin×mol⁻¹.

FIGS. 5A-5B shows analysis of entropy fluctuations on placenta tissue from TD and children with ASD. FIGS. 5A-5B carry the results of the analysis of entropy fluctuations in autism from male and female children. The analysis of outliers from TD suggests a potential failure of the feedback control of the methylation regulatory machinery on those individuals. In other words, the analysis of entropy fluctuation unveils the existence of unknown clinical condition under developing in supposedly “healthy” individuals.

FIG. 5A relates to male children. The range of entropy fluctuations in TD samples is highlighted by the horizontal hatched band.

FIG. 5B relates to female children. For visual comparison purposes the horizontal hatched band in FIG. 5B was set to cover the same range as in FIG. 5A (males). Theoretically, an ideal (healthy) feedback control of methylation regulatory machinery should keep the random fluctuation of methylation entropy very close to 1 (closer the better).

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

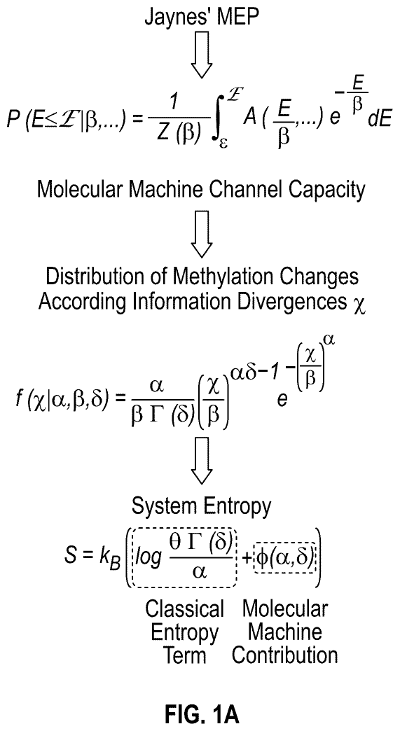

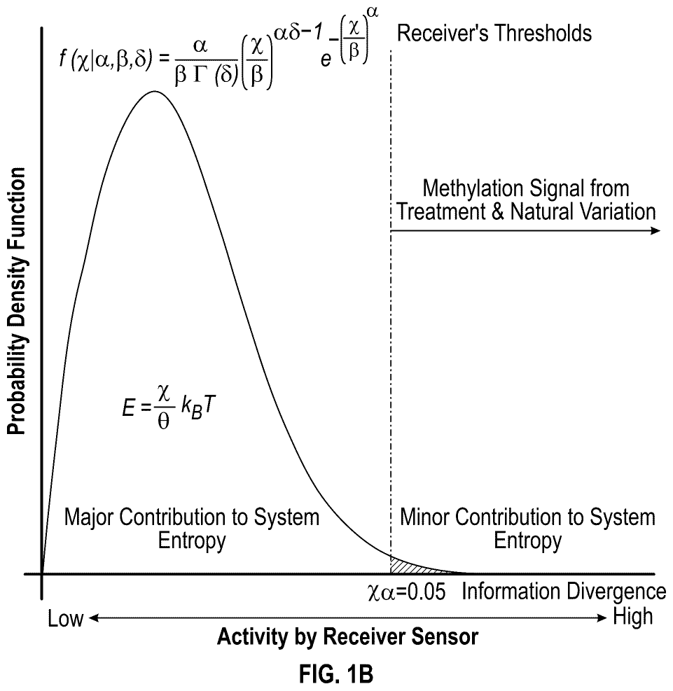

A graphical summary of the theoretical approach in the current work is shown in FIG. 1. As diagrammed, essential knowledge of the natural variability of methylation signal in control and treatment populations derives from their probability distributions. DNA methylation dynamics, as a biological system, obey thermodynamic principles. In biochemical terms, DNA methylation changes are biochemical reactions accomplished by two types of enzymes: methyltransferases and demethylases. These enzymes, as molecular machines, accomplish methylation changes through several steps of logical operations, each requiring, in accordance with Landauer's principle, a minimum energy dissipation ε=k_{B}T ln 2 per bit of information per machine operation; at the temperature of the human body, 310.15 K: ε=1.784 J·mol⁻¹. Thus, in accordance with Landauer's principle, any methylation change involves an associated amount of energy dissipation E≥k_{B}T ln 2 per bit of information per machine operation, where k_{B} stands for Boltzmann constant and T stands for the absolute temperature.

### Statistical-Physical Modeling of the Methylation Background Process

The most probable distribution of methylation states on a DNA molecule driven by spontaneous/random fluctuations can be obtained by maximizing the thermodynamic entropy under general system constraints: i) Σᵢπᵢ=1 and ii) ΣᵢπᵢEᵢ=<E>, where πᵢ is the (discrete) probability to observe dissipation of the energy value Eᵢ, and <E> is the mathematical expectation of E. Under these assumptions, Jaynes' Maximum Entropy Principal (MEP) leads to Boltzmann distribution as the most probable distribution of the system. Assuming that the energies Eᵢ dissipated to reach the states i of the system are virtually a continuum, with some density

$$A ( \frac{E}{β} , … )$$

of states of methylation changes and energies dissipated E, the probability to observe a genome-wide energy dissipation between 0 and E can be estimated as:

$$P ( E \leq 𝔼 \vert β , … ) = \frac{1}{Z ( β , … )} {\int }_{ε}^{𝔼} A ( \frac{E}{β} , … ) {e}^{- \frac{E}{β}} d E ,$$

where

$$Z ( β , … ) = {\int }_{ε}^{∞} A ( E , β , … ) {e}^{- \frac{E}{β}}$$

dE stands for the partition function of the system and β=k_{B}T is a scaling constant. That is, the number of methylation changes per unit energy at E (A(E, β, . . . )dE) is the number of methylation changes with energies dissipated per bit of information in the infinitesimal range E to E+dE. In the probability to observe a genome-wide energy dissipation between 0 and E, the expression under the integral together with the partition function is, by definition, a probability density function denoted as:

$$f ( E \vert β , … ) = \frac{1}{Z ( β , … )} A ( E , β , … ) {e}^{- \frac{E}{β}} .$$

Notice that for A(E, β, . . . )=1, the last equation reduces to the classical expression for Boltzmann distribution. The probability density function is a general probabilistic model of the methylation background process that conforms to an exponential decay law. According to the probability density function, it is expected that for any particular case of ƒ(E|β, . . . ), the probability to observe a methylation change will decline with the increment of the amount of energy dissipated per bit of information processed by the molecular machines (enzymes). In the following sections, information-thermodynamic constraints on the molecular methylation machinery permit a maximum likelihood estimation of particular cases of function ƒ(E|β, . . . ).

### The Channel Capacity of the Methylation Machinery

A fundamental constraint to deriving the probability density function of DNA methylation changes involves the physics of information in molecular machine operations. The machine capacity is closely related to Shannon's channel capacity, the maximum amount of information that a molecular machine can gain per operation. Following Schneider, the machine capacity is bounded by:

$$C = {d}_{s p a c e} {log}_{2} \frac{{P}_{y} + {N}_{y}}{{N}_{y}} ,$$

where P_{y} is the energy dissipated by the molecular machine, N_{y} is energy of the thermal noise, and d_{space} stands for the number of independently moving parts of a molecular machine that are involved in the operation.

Following Shannon, received signals have an energy average E_{y}=P_{y}+N_{y} and E⁰=N_{y} to denote the energy dissipated with probability=1 and d_{space}=v−1 to arrive at

$${C}_{v} = ( v - 1 ) {log}_{2} \frac{{E}_{y}}{{E}^{0}} ( v = α δ ) ,$$

which implies:

$$( v - 1 ) {log}_{2} \frac{{E}_{i}}{{E}^{0}} \leq {C}_{v} .$$

### Derivation of the Probability Density Function of the Methylation Background

Probability density functions (PDFs) are searched from the generalized gamma (GG) distribution family. The following assumptions must hold:
Let Nᵢ be the number of times that an amount of energy E in the interval [Eᵢ₋₁, Eᵢ) is dissipated.
Let us consider a number k of such events: N₁, N₂, . . . , Nₖ, where Σᵢ^{k} Nᵢ=N.
Let pᵢ be the probability that an amount of energy E is dissipated in the interval [Eᵢ₋₁, Eᵢ), where Σᵢ^{k} pᵢ=1.

The number of ways a set {Nᵢ|i=0, . . . , k} can be realized in a sequence of length N is given by the multinomial coefficient:

$$\frac{N !}{{N}_{1} ! \times {N}_{2} ! \times … \times {N}_{k} !}$$

and the probability of each sequence of events is: p₁^{N}^{₁}×p₂^{N}^{₂}× . . . ×pₖ^{N}^{ₖ}. Hence the probability that N distinguishable methylations events result in N₁ outcomes with energy dissipated in the interval [E₀, E₁), N₂ outcomes with energy dissipated in the interval [E₁, E₂), . . . , and Nₖ outcomes in the interval [Eₖ₋₁, Eₖ) is given by the multinomial distribution:

$$P ( {N}_{1} , … , {N}_{k} , N , {p}_{1} , … , {p}_{k} ) = \frac{N !}{{\prod }_{i = 1}^{k} {N}_{i} !} {\prod }_{i = 1}^{k} {p}_{i}^{{N}_{i}} !$$

Thus, the most probable distribution of methylation states in the system (DNA molecule) is determined by the set of values {Nᵢ} and {pᵢ}, and the constant N, which in the current case is number of cytosine sites in the DNA molecule. Continuous action of the Second Law of Thermodynamics is expected to maximize the Boltzmann entropy inside each cell, which, in turns, leads to the most probable density of methylation states. Hence, the values \hat{N}ᵢ of Nᵢ that maximize the probability P for a fixed set of probability values {pᵢ}. In particular, the following requirements are imposed on pᵢ and Nᵢ, which for the sake of better comprehension are repeated here:
1) probabilities pᵢ are proportional to a specific power of the energies Eᵢ:

$${p}_{i} = {( \frac{{E}_{i}}{{E}^{0}} )}^{v - 1}$$

where E⁰ stands for the energy dissipated with probability 1.
2) for each choice of α the following sum is a positive constant:

$${\sum }_{i = 1}^{k} {N}_{i} {E}_{i}^{α} = {E}_{const}$$

where E>0; Nᵢ's are assumed large numbers.

This problem is solved by writing the Lagrangian [symbol] =(N₁, . . . , Nₖ, β, λ), which consists of a linear combination of the logarithm of P with the relevant constraints:

$$ℒ = log N ! + {\sum }_{i = 1}^{k} {N}_{i} ( v - 1 ) log \frac{{E}_{i}}{{E}^{0}} - {\sum }_{i = 1}^{k} log {N}_{i} ! - \frac{1}{λ} ( {\sum }_{i = 1}^{k} {N}_{i} {E}_{i}^{α} - {E}_{const} ) - μ ( {\sum }_{i = 1}^{k} {N}_{i} - N )$$

After approximating the factorial for large N using Stirling's formula and setting the derivatives

$$\frac{\partial ℒ}{\partial {N}_{i}} = 0 ( i = 0 , … , k ) ,$$

this yields the equations:

$$( v - 1 ) log \frac{{E}_{i}}{{E}^{0}} - log {N}_{i} ! - \frac{{E}_{i}^{α}}{λ} - μ = 0$$

Which, after setting η=e^{-μ}(λ/E⁰)^{v-1}, leads to the expressions:

$${\hat{N}}_{i} = η {( \frac{{E}_{i}}{λ} )}^{v - 1} {e}^{- \frac{{E}_{i}^{α}}{λ}}$$

$$and$$

$$N = η {\sum }_{i}^{N} {( \frac{{E}_{i}}{λ} )}^{v - 1} {e}^{- \frac{{E}_{i}^{α}}{λ}}$$

The last equation permits us to update the discrete probability distribution πᵢ under assumptions 1 (probabilities pᵢ are proportional to a specific power of the energies Eᵢ) and 2 (for each choice of α the following sum is a positive constant):

$${π}_{i} = \frac{{\hat{N}}_{i}}{N} = \frac{{( \frac{{E}_{i}}{λ} )}^{v - 1} {e}^{- {( \frac{{E}_{i}}{λ} )}^{α}}}{{\sum }_{i}^{N} {( \frac{{E}_{i}}{λ} )}^{v - 1} {e}^{- {( \frac{{E}_{i}}{λ} )}^{α}}}$$

That is, \hat{N}ⁱ/N can be interpreted as discrete probability distribution associated with the events of energy dissipation under the system constraints 1 and 2. Alternatively, the equation above can be written as:

$$\frac{{\hat{N}}_{i}}{N} = Δ {E [ {\sum }_{i = 0}^{k} {( \frac{{E}_{i}}{λ} )}^{v - 1} {e}^{- {( \frac{{E}_{i}}{λ} )}^{α}} Δ E ]}^{- 1} {( \frac{{E}_{i}}{λ} )}^{v - 1} {e}^{- \frac{{E}_{i}^{α}}{λ}}$$

which after setting λ=β^{α} and δ=v/α can be rewritten as:

$$\frac{{\hat{N}}_{i}}{N} = Δ {E [ {\sum }_{i = 0}^{k} {( \frac{{E}_{i}}{β} )}^{αδ - 1} {e}^{- {( \frac{{E}_{i}}{β} )}^{α}} Δ E ]}^{- 1} {( \frac{{E}_{i}}{β} )}^{αδ - 1} {e}^{- {( \frac{{E}_{i}}{β} )}^{α}}$$

Which after setting k→∞, the size of the interval ΔE→0 and the sum in the bracketed coefficient can be replaced by a definite integral that yields:

$${\int }_{0}^{∞} {( \frac{{E}_{i}}{β} )}^{αδ - 1} {e}^{- {( \frac{{E}_{i}}{β} )}^{α}} dE = \frac{β Γ ( δ )}{α} = Z$$

and thus:

$$\frac{{\hat{N}}_{i}}{N} = Δ E \frac{α}{β Γ ( δ )} {( \frac{{E}_{i}}{β} )}^{αδ - 1} {e}^{- {( \frac{{E}_{i}}{β} )}^{α}}$$

Hence, the integration from 0 to E of the right side of the equation above satisfies the equation that determines the probability to observe a genome-wide energy dissipation between 0 and E. After assuming that the energies Eᵢ dissipated to reach the states i of the system are virtually a continuum, probability density function denoted as ƒ(E|β, . . . ) becomes the generalized gamma probability density function ƒ(E|β, δ).

### Probability Density Function of the Methylation Background Changes

The probability to observe a genome-wide energy dissipation between 0 and E and probability density function quantitatively summarize the statistical physics underlying methylation changes that are not induced by the methylation regulatory machinery. Application of thermodynamic principles to chromatin dynamics tends to maximize Boltzmann entropy, leading, in turn, to the most probable methylation density states. The probability sought to be maximized: P=(N₁, . . . , Nₖ, N, p₁, . . . , pₖ) that N distinguishable methylation events result in N₁, . . . , Nₖ (Σᵢ^{k} Nᵢ=N) outcomes in the intervals [E₁, E₂), . . . , [Eₖ₋₁, Eₖ) with probabilities p₁, . . . , pₖ.

Two basic assumptions were imposed on pᵢ, Nᵢ and Eᵢ:
1) probabilities pᵢ are proportional to a specific power of the energies Eᵢ:

$${p}_{i} = {( \frac{{E}_{i}}{{E}^{0}} )}^{v - 1}$$

2) for each choice of α the following sum is a positive constant:

$${\sum }_{i = 1}^{k} {N}_{i} {E}_{i}^{α} = {E}_{const}$$

where E>0; Nᵢ's are assumed large numbers.

The first assumption derives from a current interpretation of the channel capacity of molecular machines as given by the channel capacity of the methylation machinery. The second assumption implies that parameter a carries information about the molecular machine, since v=αδ. A maximum likelihood estimation of function ƒ(E|β, . . . ), on a thermodynamic basis, adapting the Lienhard and Meyer approach to the specific scenario of DNA methylation, is provided in the subsection: Derivation of the probability density function of the methylation background, supra. The above assumptions lead to the generalized gamma probability density function:

$$f ( E \vert β , δ ) = \frac{α}{βΓ ( δ )} {( \frac{E}{β} )}^{αδ - 1} {e}^{- {( \frac{E}{β} )}^{α}}$$

where α>0, β>0, δ>0, and E>0. Consistently with the probability density function, the analytical expression for partition function derives from the generalized gamma probability density function:

$$Z ( β ) = {\int }_{0}^{∞} {( \frac{E}{β} )}^{αδ - 1} {e}^{- {( \frac{E}{β} )}^{α}} dE = \frac{β Γ ( δ )}{α} .$$

Hence, the density A(E, β, . . . ) can be expressed as:

$$A ( E , β , δ ) = {( \frac{E}{β} )}^{αδ - 1} {e}^{- {( \frac{E}{β} )}^{α - 1}} .$$

An information-theoretic divergence χ(p, q) of methylation levels p and q will follow a distribution derived from the probability to observe a genome-wide energy dissipation between 0 and E (Generalized Gamma, Gamma, or Weibull distribution model), provided that it is proportional to the energy E. In this case, the energy dissipated E is per bit of information associated to the corresponding methylation changes. For an information-theoretic divergence measure of methylation levels χ(p, q), the same analytical steps are followed to derive the generalized gamma probability density function (see the subsection: Derivation of the probability density function of the methylation background, supra), leading to a probability density function for the information divergence χ(p, q):

$$f ( χ \vert α , θ , ψ ) = \frac{α}{θ Γ ( δ )} {( \frac{χ}{θ} )}^{αδ - 1} {e}^{- {( \frac{χ}{θ} )}^{α}} .$$

Assuming that

$$\frac{{E}_{t}}{{k}_{B}} = \frac{χ}{θ}$$

(χ in bit units), the energy dissipated can be estimated as:

$$E = \frac{χ}{θ} {k}_{B} T .$$

For a molecular machine working under ideal conditions, the minimum energy dissipated is, according to Landauer's principle, E=χk_{B}T ln 2, or, θ=1/ln 2 in ideal conditions. A more general distribution including the location parameter μ is given as:

$$f ( χ \vert α , θ , μ , δ ) = \frac{α}{θ Γ ( δ )} {( \frac{χ - μ}{θ} )}^{αδ - 1} {e}^{- {( \frac{χ - μ}{θ} )}^{α}}$$

with mean:

$$v = \frac{μ Γ ( δ ) + θ Γ ( δ + \frac{1}{α} )}{Γ ( δ )}$$

and variance:

$$σ = \frac{{μ}^{2} Γ ( δ ) + 2 μ θ Γ ( \frac{1}{α} + δ ) + {θ}^{2} Γ ( \frac{2}{α} + δ )}{Γ ( δ )} .$$

In particular, χ(p, q) can be expressed in terms of the Hellinger divergence given by Sanchez et al., “Discrimination of DNA Methylation Signal from Background Variation for Clinical Diagnostics”, or in terms of J-divergence. The most frequent members of a general gamma distribution family found by goodness-of-fit tests for processed bisulfite sequence datasets from different species are Weibull (δ=1) and Gamma (α=1) distributions, obtained as particular cases from the generalized gamma probability density function. See Sanchez et al., “Integrative Network Analysis of Differentially Methylated and Expressed Genes for Biomarker Identification in Leukemia”, Sci. Rep. 10, 2123 (2020), herein incorporated by reference in its entirety.

A Connection with Shannon's Communication Theory

As suggested by the present inventors in previous report(s), genome-wide patterning of cytosine DNA methylation occurs at specific landmarks, statistically alluding to the existence of a sort of methylation language/code. See e.g., Sanchez et al., “Genome-wide discriminatory information patterns of cytosine DNA methylation”, Int. J. Mol. Sci. 17, 938 (2016), which is hereby incorporated by reference in its entirety herein. In terms of Shannon's communication theory, a communication system can be described by the conditional probability (density) Pₓ(y), so that if message x is produced by the source, the recovered message at the receiving point will be y. Shannon defined the rate R₁ of generating information for a given quality v₁=∫∫ρ(x, y)P(x, y) dx dy of reproduction to be

$$R = {min}_{{P}_{x}} \int \int P ( x , y ) log \frac{P ( x , y )}{P ( x ) P ( y )} dx dy$$

at fixed v₁ and variable Pₓ(y), where ρ(x, y) is a distance function. Shannon showed that function ρ(x, y) behaves as a “distance” between x and y to measure the unlikelihood, based on a fidelity criterion, to receive y with transmission of x.

In Shannon's analysis, the conditional probability P_{y}(x) that minimizes the rate R is given by the expression P_{y}(x)=B(x)e^{-λρ(x,y)}, where B(x) is chosen to satisfy ∫B(x)e^{-λρ(x,y)} dx=1. In function B(x), the transmitted message x can be expressed at each cytosine site in terms of observed methylation levels in a treatment or a patient group. Methylation levels are estimated as: nCᵢ^{m}/(nC^{m}+nCᵢ), where nCᵢ^{m} and nCᵢ are the number of times that the cytosine is observed methylated and unmethylated at site i, respectively. The received message y can be specified as reference methylation levels, which could be the centroid of a group control or estimated from an independent subset of control samples from a control population. Next, function ρ(x, y) can be expressed in terms of a symmetric information divergence χ(x, y) between the methylation levels x and y. For a fixed reference y, the equality χ(x, y)= (x) makes it possible to choose B(x) as:

$$B ( x ) = {χ}^{′} ( x ) \frac{α}{θ Γ ( δ )} {( \frac{χ ( x )}{θ} )}^{αδ - 1} {e}^{- {( \frac{χ ( x )}{θ} )}^{α - 1}} .$$

Where dχ=χ′(x) dx and λ=1/θ. The conditional probability P_{y}(x) that, if the recovered message at the receiving point is y, the original message produced by the source is x, can be reinterpreted (after the change of variables) as:

$${P}_{y} ( χ \vert α , δ , θ ) = {\int }_{0}^{χ} \frac{α}{θ Γ ( δ )} {( \frac{χ}{θ} )}^{αδ - 1} {e}^{- {( \frac{χ}{θ} )}^{α}} d χ .$$

This equation indicates the probability that, if the recovered message at the receiving point is y, the information divergence between y and the original message x produced by the source is χ. This application of Shannon's reasonings leads to the following. If the organismal methylation system conforms to a communication system, then the methylation messaging, with best encoding, is described by P_{y}(χ∥α, δ, θ). This implies that if the methylation signals are messages, given in a framework of a communication system, then the probability properly describe the methylation messaging and, consequently, all the knowledge derived from it is not a mathematical artefact.

### The Gibbs Entropy of the System

The Gibbs entropy of the system, resulting from methylation changes, is defined by the integral:

$$S = - {k}_{B} {\int }_{0}^{∞} f ( E \vert α , β , δ ) ( ln f ( E \vert α , β , δ ) dE ) ,$$

which yields the known analytical expression (see the Material and Methods used to compute results presented in Table 1, infra):

$$S = {k}_{B} ( ln \frac{β Γ ( δ )}{α} + ψ ( δ ) ( \frac{1}{α} - δ ) + δ ) ,$$

where

$$ψ ( δ ) = \frac{d ln Γ ( δ )}{d δ}$$

stands for the digamma function. After considering the generalized gamma probability density function, it can be written:

$$S = {k}_{B} ln \frac{β Γ ( δ )}{α} + {k}_{B} ψ ( δ ) ( \frac{1}{α} - δ ) + {k}_{B} δ .$$

where

$$S = {k}_{B} ln \frac{β Γ ( δ )}{α}$$

is the classical entropy term and

$${k}_{B} ψ ( δ ) ( \frac{1}{α} - δ ) + {k}_{B} δ$$

is the molecular machine moving parts contribution.

Thus, the entropy of the individual methylation system splits into a classical term and the contribution from molecular machine moving parts:

$$S = {S}_{classic} + {S}_{m a c h i n e} .$$

A rough estimate of Gibbs entropy for different tissues/cells can be based on the information divergence χᵢ after expressing the energy Eᵢ in terms of χᵢ according to the probability density function for the information divergence:

$$S = {k}_{B} ( ln \frac{θ Γ ( δ )}{α} + ϕ ( α , δ ) ) ,$$

where the term

$$ϕ ( α , δ ) = ψ ( δ ) ( \frac{1}{α} - δ ) + δ$$

is a function of a model parameter associated to the number of independent moving parts of the molecular machine (v=αδ).

Since log₂ x=ln x/ln 2, the Gibbs entropy for different tissues/cells can be written as:

$$S = {k}_{B} ( {log}_{2} \frac{θ Γ ( δ )}{α} + \frac{ϕ ( α , δ )}{ln 2} ) .$$

The terms between brackets from the Gibbs entropy S (at constant temperature) correspond to Shannon entropy H, which, in this case, depends only on distribution parameters estimated from the experimental data for each individual. Thus, the Shannon entropy H can be written as:

$$H = {log}_{2} \frac{θ Γ ( δ )}{α} + \frac{ϕ ( α , δ )}{ln 2}$$

$$and$$

$$S = {k}_{B} ln 2 H .$$

Following Schneider, for a decrease in methylome entropy:

$$Δ S = {S}_{after} - {S}_{before} ,$$

there is a corresponding decrease in the uncertainty of genome-wide methylation changes:

$$Δ H = {H}_{after} - {H}_{before} .$$

Following a decrease in this uncertainty, the methylome gains information Iₘ:

$${I}_{m} = - Δ H$$

expressed in Joule per Kelvin as:

$${I}_{m} ≡ - {k}_{B} ln 2 Δ H .$$

Thus, information-theoretic entropy and thermodynamic entropy yield identical outcomes, up to the product of Boltzmann's constant by ln 2, even though they are independent functions.

If Iₘ>0, then the methylation system experienced a gain of information, otherwise, if Iₘ<0, the system experienced a loss of information.

### Thermodynamic Potential of Methylation Changes

Assuming that a balance exists between methylation and demethylation processes along each DNA molecule, the overall mass (the number of molecules N) and volume (V) of the DNA molecule remain constant. This assumption holds in most experimental datasets since, for sufficiently large genomic regions, the sum of the difference in methylation level is close to zero. Under this condition, and assuming constant temperature (T), methylation changes on a DNA molecule and the micro-environment around them can be treated as a closed system to mass transport but not energy transfer. In statistical physics, these systems are referred to as NVT systems, with the thermodynamic variables N, V, and T held fixed. Helmholtz free energy (F) represents the driving force for NVT systems, the thermodynamic potential that measures “useful” work obtainable from a closed system at a constant temperature and volume.

Helmholtz free energy can be estimated from its definition: F=U−TS. Assuming that the molecular machine operations do not change the internal energy U of the system, this becomes: ΔF=−TΔS or

$$Δ F = - β ( ln \frac{β Γ ( δ )}{α} + ψ ( δ ) ( \frac{1}{a} - δ ) + δ )$$

The same result derives from the Gibbs free energy definition: G=H−TS. Given that the molecular machine operations do not change the system pressure, (ΔH=0): ΔG=−TΔS, the change in Helmholtz free energy roughly estimates how much Helmholtz free energy would be involved in methylation. Rough estimations based on the information divergence χ can use the approach:

$$Δ F = - β ( ln \frac{θ Γ ( δ )}{α} + ϕ ( α , δ ) ) .$$

where β=k_{B}T. From the entropy of the individual methylation system, the Helmholtz free energy can be split into the classical term and the contribution of molecular machine moving part:

$$Δ F = - β {S}_{classic} - β {S}_{m a c h i n e} = Δ {F}_{classic} + Δ {F}_{m a c h ine} ,$$

where according with the analytical expression for partition function:

$$Δ {F}_{classic} = {k}_{B} T ln \frac{θ Γ ( δ )}{a} = {k}_{B} T ln Z .$$

The particular cases of S_{G} and F(β) for Weibull and Gamma distributions are obtained with parameter values δ=1 and α=1, respectively. Substitution of the Gibbs entropy for different tissues/cells in the change in Helmholtz free energy yields:

$$Δ F = - β ln 2 H .$$

At constant temperature, ΔF decreases with the increment of Shannon entropy in the system. The variation of Helmholtz free energy between two system states (before and after) can be expressed as:

$$ΔΔ F = Δ {F}_{after} - Δ {F}_{before}$$

or, considering the methylome gains information:

ΔΔF=TIₘ

where a loss of information (Iₘ<0) will be associated with loss of free energy ΔΔF<0.

### Biological Applications

Entropy is a thermodynamic state variable of the system, which means that its value is completely determined by the current state of the system and not by how the system reached that state. Therefore, the interest is in learning whether the entropy of the methylation system, measured with respect to a reference state, coincides with an observable phenotypic change. Functions for entropy and Helmholtz free energy estimations, given by the rough estimate of Gibbs entropy for different tissues/cells and the change in Helmholtz free energy, respectively, are included in the MethylIT platform (R package). Entropy was estimated in Arabidopsis thaliana Col-0 accessions (wild type control, WT) grown under two different growth conditions, the methyltransferase mutant met1, and a sequential, six-generation, single-seed decent population of msh1-derived heritable epigenetic memory (mm) and full-sib non-memory (nm) lines.

In Arabidopsis, seedlings grown in vitro versus in soil pots under short versus long daylength can experience dramatic effects on plant growth. We, therefore, compare methylome outcomes from wild type Col-0 2-week-old seedling growth on nutrient media at 24-hr daylength and mature plant growth on potting media at 12-hr daylength. CG methylation in plants is maintained by METHYLTRANSFERSE1 (MET1) and mutations that disrupt its activity induce genome-wide CG hypomethylation. Data from this mutant to test is used for observable loss of information in met1 plants relative to wild type grown under the same conditions (34). In the case of msh1 memory state, heritable epigenetic stress memory occurs following RNAi suppression of the MutS HOMOLOG (MUTS) gene in Arabidopsis, yielding ca. 20% of the RNAi transgene-null progeny with a heritable memory phenotype of delayed maturation and sustained stress response (mm), and the remainder appearing unchanged in phenotype and designated “non-memory” (nm). A six-generation lineage of msh1 memory was described previously, and both generation-1 memory (mm1) and non-memory (nm1) full-sib types display evidence of genome-wide cytosine methylation repatterning relative to wild type. Here, an analysis of samples is included from the six-generation msh1 memory lineage and predict these variants to display a more incremental effect on entropy variation than the met1 mutant. Results shown in Table 1 for generation 1 (mm1, nm1) and generation 3 (mm3) confirm these predicted outcomes.

TABLE 1
Gibbs entropy estimated in Arabidopsis msh1 epigenetic memory (mm1, mm3),
nonmemory (nm), met1 mutant and corresponding Col-0 controls (WT).

| | Gibbs entropy by individual chromosome | | | | | | |
|---|---|---|---|---|---|---|---|
| Treatment | 1 | 2 | 3 | 4 | 5 | Iₘ | ΔΔF |
| WT3-1 | −12.095 | −13.092 | −12.854 | −12.875 | −12.398 | | |
| WT3-2 | −12.239 | −13.202 | −12.827 | −12.955 | −12.447 | | |
| WT3-3 | −12.582 | −13.611 | −13.312 | −13.403 | −12.872 | | |
| WT3-4 | −12.190 | −13.289 | −12.884 | −13.008 | −12.534 | | |
| WT3-5 | −13.010 | −14.074 | −13.806 | −13.831 | −13.333 | | |
| nm1_1 | −10.517 | −11.671 | −11.43 | −11.447 | −10.970 | −0.612**† | −189.8**† |
| nm1_2 | −10.344 | −11.461 | −11.193 | −11.205 | −10.758 | | |
| nm1_3 | −13.424 | −14.234 | −14.126 | −14.175 | −13.761 | | |
| nm1_4 | −10.332 | −11.428 | −11.16 | −11.192 | −10.74 | | |
| nm1_5 | −14.458 | −14.972 | −15.002 | −14.804 | −14.614 | | |
| mm1_1 | −12.452 | −13.385 | −13.153 | −13.134 | −12.807 | −1.140*** | −353.63*** |
| mm1_2 | −13.170 | −14.111 | −13.934 | −13.978 | −13.579 | | |
| mm1_3 | −10.485 | −11.578 | −11.391 | −11.369 | −10.947 | | |
| mm1_4 | −10.087 | −11.177 | −10.972 | −10.982 | −10.485 | | |
| mm1_5 | −9.969 | −11.104 | −10.818 | −10.852 | −10.298 | | |
| mm3_1 | −9.504 | −10.593 | −10.366 | −10.370 | −9.850 | −2.627*** | −814.79*** |
| mm3_2 | −9.617 | −10.691 | −10.537 | −10.528 | −10.014 | | |
| mm3_3 | −9.392 | −10.475 | −10.269 | −10.264 | −9.839 | | |
| mm3_4 | −10.336 | −11.407 | −11.292 | −11.310 | −10.825 | | |
| mm3_5 | −9.688 | −10.736 | −10.531 | −10.526 | −10.083 | | |
| WTₘₑₜ₁_{_{—}}1 | −3.751 | −4.061 | −3.958 | −3.738 | −3.700 | | |
| WTₘₑₜ₁_{_{—}}2 | −5.876 | −6.242 | −6.164 | −5.959 | −5.811 | | |
| WTₘₑₜ₁_{_{—}}3 | −5.869 | −6.216 | −6.070 | −5.896 | −5.727 | | |
| WTₘₑₜ₁_{_{—}}4 | −5.994 | −6.347 | −6.178 | −5.995 | −5.889 | | |
| met1_1 | 2.183 | 2.129 | 2.065 | 1.980 | 2.085 | −7.185*** | −2228.45*** |
| met1_2 | 1.199 | 1.126 | 1.072 | 1.004 | 1.108 | | |
| met1_3 | 2.032 | 1.993 | 1.923 | 1.848 | 1.946 | | |

Entropy values were estimated using Gibbs entropy S and J-divergence. The values are given in J × K⁻¹ × mol⁻¹, after replacing Boltzmann constant by the Gas constant. Loss of Information (Iₘ) is given by Iₘ = −k_{B} ln 2 ΔH. Helmholtz free energy (ΔΔF) values were estimated using ΔΔF = TIₘ and J-divergence. The values are given in kJ × mol⁻¹.
Symbols ‘**’ and ‘***’ indicate highly statistically significant differences at p-value < 0.01 and p-value < 0.001 between mutant or memory state, respectively.
Symbol † indicates Wilcoxon paired test, otherwise testing was conducted applying linear mixed model.

Entropy values were estimated using Gibbs entropy S and J-divergence. The values are given in J×K⁻¹×mol⁻¹, after replacing Boltzmann constant by the Gas constant. Loss of Information (Iₘ) is given by Iₘ=−k_{B} ln 2 AH. Helmholtz free energy (ΔΔF) values were estimated using ΔΔF=TIₘ and J-divergence. The values are given in kJ×mol⁻¹. Symbols ‘**’ and ‘***’ indicate highly statistically significant differences at p-value <0.01 and p-value <0.001 between mutant or memory state, respectively. Symbol † indicates Wilcoxon paired test, otherwise testing was conducted applying linear mixed model.

The effect of an msh1 suppression line on genome-wide methylation changes in epigenetic memory and non-memory progeny was reflected in a discrete increment of entropy and, consequently, loss of information: ΔS=S_{control}−Sₘᵤₜₐₙₜ<0, i.e., Iₘ<0. This observation is further evidence of the epigenetic effects that give rise to the memory state. A markedly greater loss of information is observed in the met1 mutant, consistent with the significant effects of genome-wide CG demethylation.

Results suggest that entropy is also a highly sensitive measure of the state of an organism. There are significant differences in the entropy values for Col-0 wildtype controls WT3 and WTₘₑₜ₁. Although these wildtype controls derive from the same Arabidopsis accession, they differ in ontogeny. WTₘₑₜ₁ plants were grown under continuous light for two weeks in half-strength Gamborg's B5 media, while WT3 plants were grown to maturity on standard peat mix in pots maintained at twelve-hour (12-hr) daylength and sampled at bolting stage. These differences in plant stage and growth conditions account for the marked entropy differences observed.

In human studies, Gibbs entropies for different cancer cells and the corresponding healthy tissue/cell controls are presented in Table 2.

TABLE 2
Gibbs entropy estimated in human cancer
cells and corresponding normal tissue.

Chromosome

| Tissue | 7 | 9 | 17 | 22 |
|---|---|---|---|---|
| Brain | −16.507 | −16.130 | −15.959 | −15.403 |
| Glioma | 3.596 | −2.006 | −2.326 | −0.970 |
| Breast | −14.559 | −14.078 | −13.606 | −12.912 |
| Breast Cancer | 2.388 | −1.271 | −4.081 | −1.465 |
| Breast | 4.729 | 3.237 | 6.448 | 2.882 |
| Metastasis | | | | |
| Colon | −14.782 | −14.379 | −14.417 | −13.965 |
| Colon Cancer | −10.088 | −10.326 | −10.712 | −10.036 |
| Colon | −5.178 | −6.442 | −7.776 | −7.686 |
| Metastasis | | | | |
| Lung | −16.735 | −16.554 | −15.969 | −15.622 |
| Lung Cancer | −8.027 | −8.520 | −7.808 | −9.854 |
| HESC 1 | 1.981 | 1.988 | 2.078 | 2.147 |
| HESC 2 | 1.645 | 1.641 | 1.787 | 1.840 |
| HESC 3 | 1.724 | 1.728 | 1.833 | 1.897 |

Energy values were estimated using S=S_{classic}+S_{machine} and J-divergence. The values are given in J×K⁻¹×mol⁻¹. Human embryonic stem cell (HESC) values are provided as reference for an undifferentiated tissue.

Outcomes suggest that Gibbs entropy increases for all cancer cells relative to their corresponding normal tissue. Since information divergences were computed with respect to the same reference individual, the observed entropy values suggest that breast metastasis cells underwent the most aggressive loss of information (assuming that experimental errors were not large enough to affect the estimated values). The relationship between Gibbs entropy and Helmholtz free energy predicts results shown in Table 3.

TABLE 3
Helmholtz free energy estimates in cancer
cells and corresponding normal tissue.

Chromosome

| Tissue | 7 | 9 | 17 | 22 |
|---|---|---|---|---|
| Brain | 5.120 | 5.003 | 4.950 | 4.777 |
| Glioma | −1.115 | 0.622 | 0.721 | 0.301 |
| Breast | 4.515 | 4.366 | 4.220 | 4.005 |
| Breast Cancer | −0.741 | 0.394 | 1.266 | 0.454 |
| Breast | −1.467 | −1.004 | −2.000 | −0.894 |
| Metastasis | | | | |
| Colon | 4.585 | 4.460 | 4.471 | 4.331 |
| Colon Cancer | 3.129 | 3.203 | 3.322 | 3.113 |
| Colon | 1.606 | 1.998 | 2.412 | 2.384 |
| Metastasis | | | | |
| Lung | 5.190 | 5.134 | 4.953 | 4.845 |
| Lung Cancer | 2.489 | 2.643 | 2.422 | 3.056 |
| HESC 1 | −0.614 | −0.617 | −0.644 | −0.666 |
| HESC 2 | −0.510 | −0.509 | −0.554 | −0.571 |
| HESC 3 | −0.535 | −0.536 | −0.569 | −0.588 |

Energy values are given in kJ×mol⁻¹. Human embryonic stem cell (HESC) values are provided as reference for an undifferentiated tissue.

After the methylation reprogramming that transforms differentiated healthy cells to a cancer state, the information potential of cancer cells appears to decrease dramatically relative to healthy tissue cells. These data reflect an important, previously undocumented, means of assessing the state of a biological system.

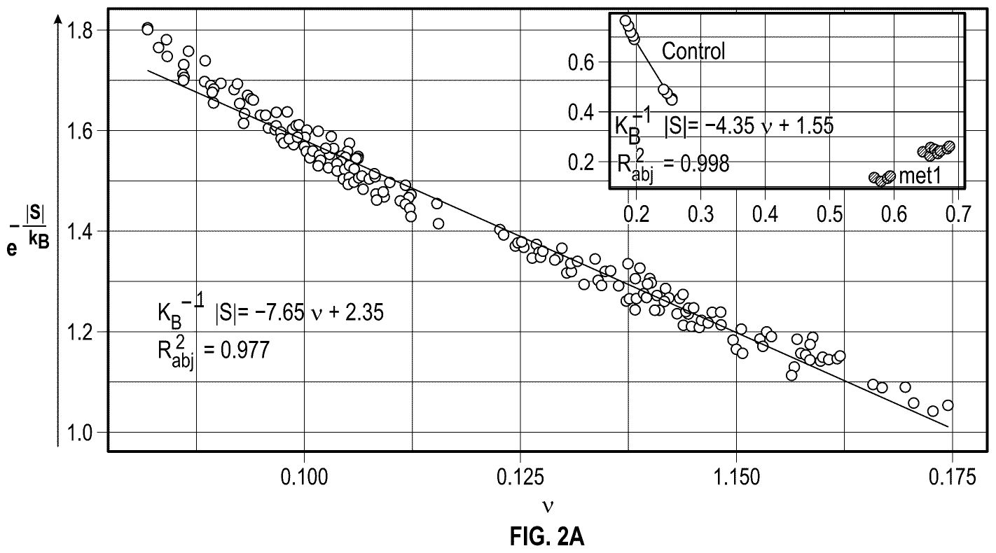

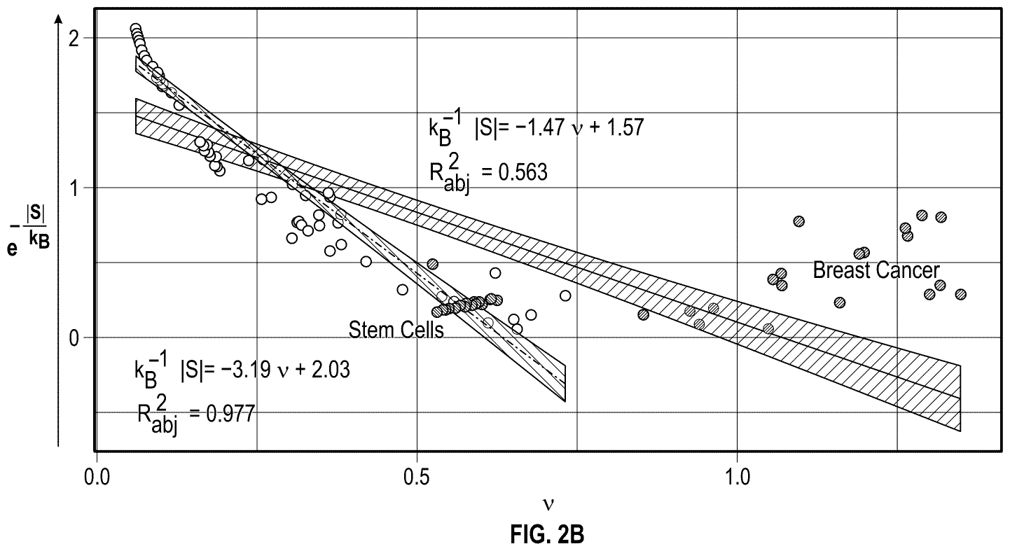

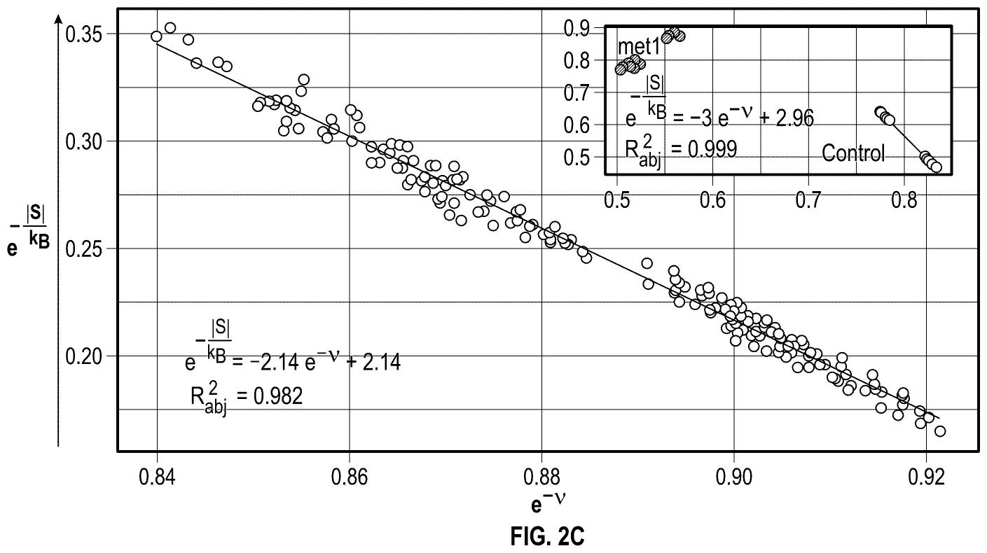

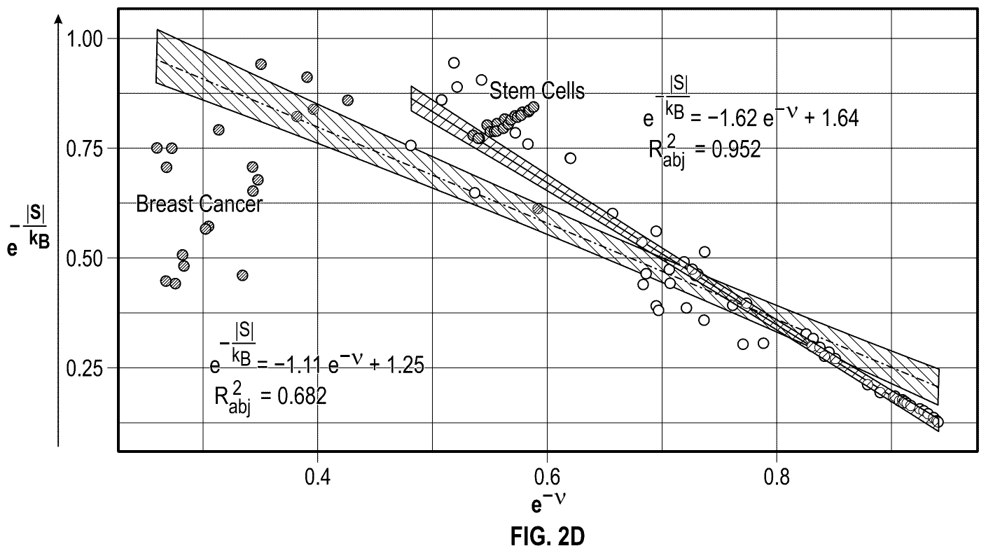

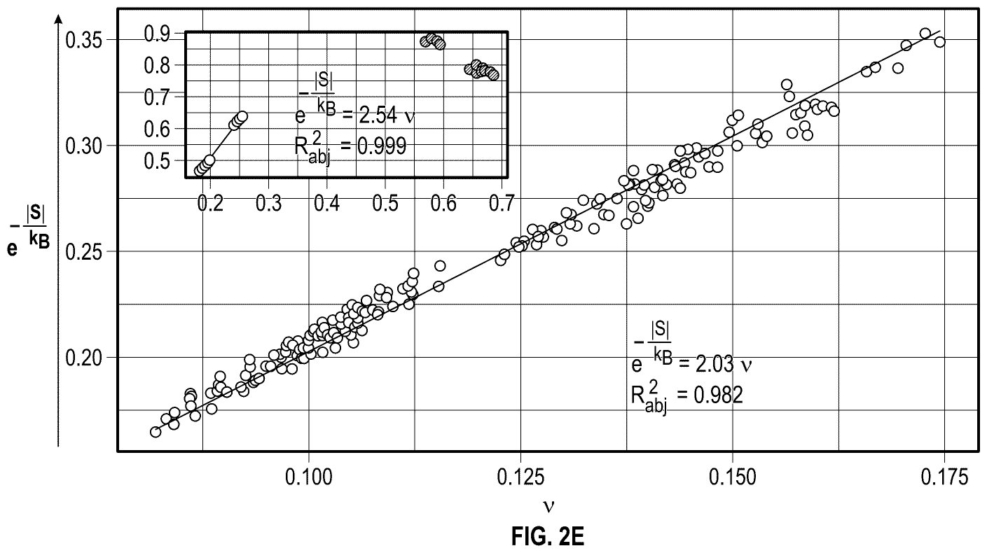

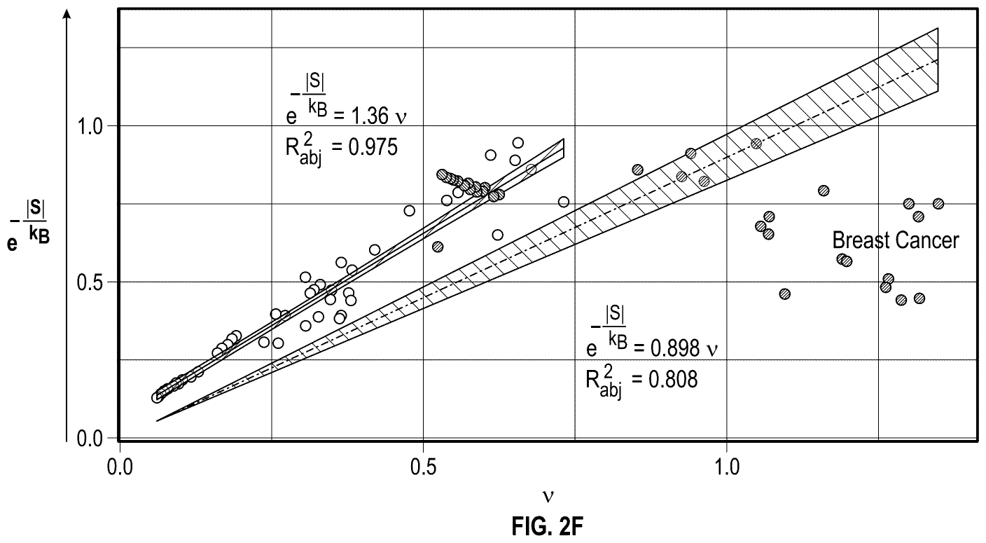

Large values v= [symbol] χ [symbol] of the information divergence χ are indicative of large amount (genome-wide) of energy dissipated by the methylation machinery. Then, the physical work accomplished by the methylation machinery must lead to a decreasing of the genome-wide methylation uncertainty, which must be reflected in the values of the (dimensionless) entropy k_{B}⁻¹S. This conjecture is supported by the regression analysis k_{B}⁻¹|S| versus v accomplished on Arabidopsis and in human datasets (FIGS. 2A-2B). The last results permit us to get an empirical estimation of the entropy fluctuations through the regression analysis e^{-k}^{_{B}}^{-1|S|} versus e^{-v} (FIGS. 2C-D), which leads to the equation:

$${e}^{- {k}_{B}^{- 1} \vert S \vert} = η ( 1 - {e}^{- v} )$$

The last equality was verified through regression analysis e^{-k}^{_{B}}^{-1|S|} versus e^{-v} in Arabidopsis and human datasets (FIGS. 2C-2D). The equation can be written as the quotient:

$$\frac{{e}^{- {k}_{B}^{- 1} \vert S \vert}}{1 - {e}^{- v}} = η ,$$

which is another way to express the fluctuation theorem in a DNA methylation context. The model parameter η is an experimentally measurable quantity that characterizes the efficacy of feedback control.

As shown in FIG. 2C and FIG. 2D, larger values of model parameter n are associated with better feedback control, which is reflected in better model fitting to the datasets.

The validity of model e^{-k}^{_{B}}^{-1|S|}=η(1−e^{-v}) implies the validity, up to the experimental error, of model e^{-k}^{_{B}}^{-1|S|}=ηv, which is derived after introducing the e^{-V}≈1−v in the initial model.

Results from the regression analyses e^{-k}^{_{B}}^{-1|S|} versus v in Arabidopsis and in human datasets support this expression (FIGS. 2E-2F). This compliance comes with exceptions for the extreme conditions found in Arabidopsis mutant met1 (points with stripe pattern in FIG. 2E, subplot), breast cancer (points with right-rotated stripe pattern) and stem cells (points with left-rotated stripe pattern, FIG. 2F).

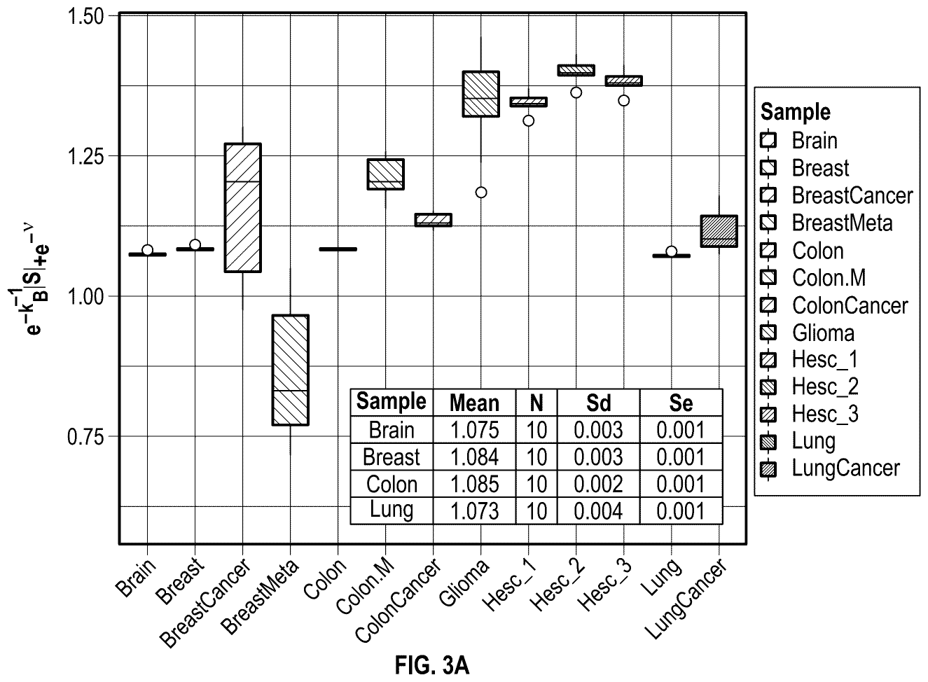

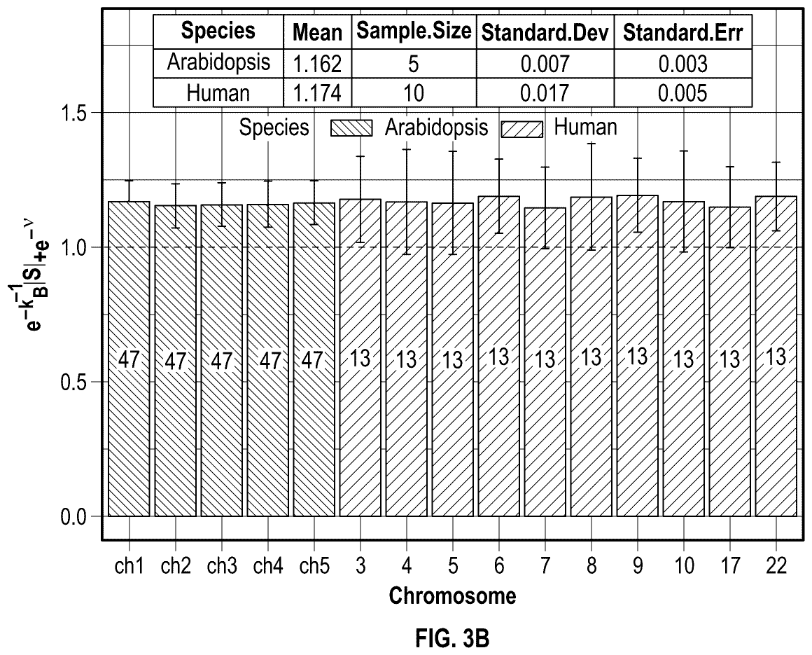

When considering the average of the sum of Boltzmann's factors e^{-k}^{_{B}}^{-1|S|} and e^{-v}, results suggested that the average sum e^{-k}^{_{B}}^{-1|S|}+e^{-v} appears constant (FIGS. 3A-B). In particular, no statistical differences between the overall means of values from Arabidopsis (FIG. 3A) and humans were found (FIG. 3B), which leads to the postulation:

$$〈 {e}^{- {k}_{B}^{- 1} \vert S \vert} + {e}^{- v} 〉 = λ$$

where λ=1 or has a value close to 1. Thus, [symbol] e^{-k}^{_{B}}^{-1|S|} [symbol] =1− [symbol] e^{-v} [symbol] can be written, and with feedback control: [symbol] e^{-k}^{_{B}}^{-1|S|} [symbol] =η(1−( [symbol] ^{-v} [symbol] ). This leads to the equation previously derived following independent reasoning path:

$${e}^{- {k}_{B}^{- 1} \vert S \vert} = η ( 1 - {e}^{- v} ) .$$

The data and the R script to build FIG. 3B are given in the EXAMPLES below.

Again, only extreme conditions did not fit the model. In biological terms, the derivation of the quotient

$$\frac{{e}^{- {k}_{B}^{- 1} \vert S \vert}}{1 - {e}^{- v}} = η$$

implies the magnitude of genome-wide methylation changes deriving from response to environmental change is restricted. FIGS. 2A-F and 3A-3B show that only extreme situations found in Arabidopsis met1 and cancer cells do not hold to restrictions.

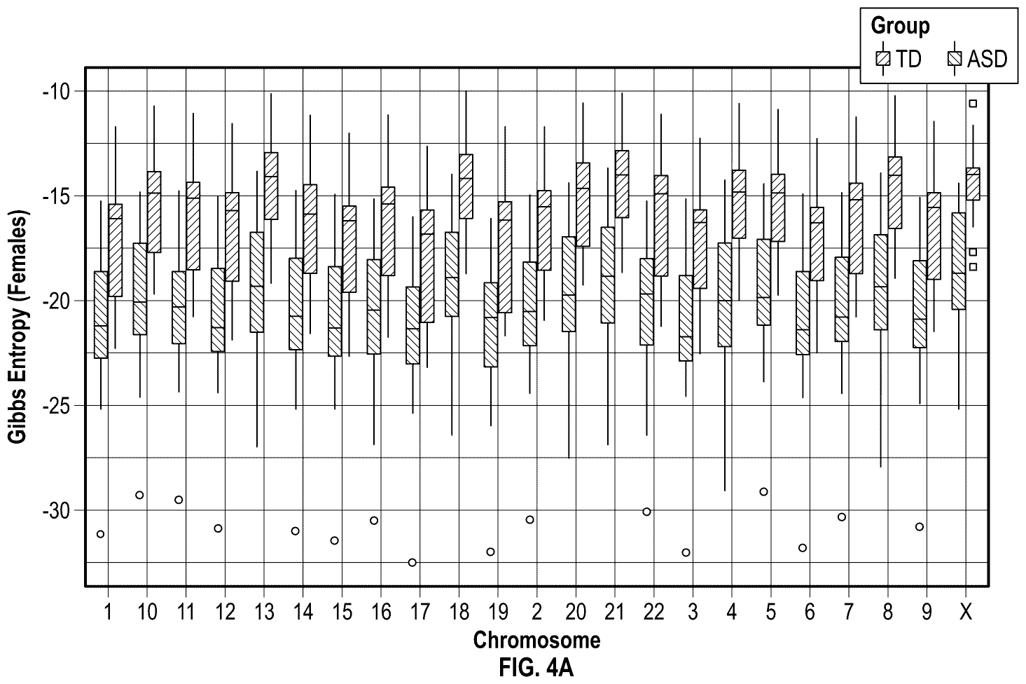

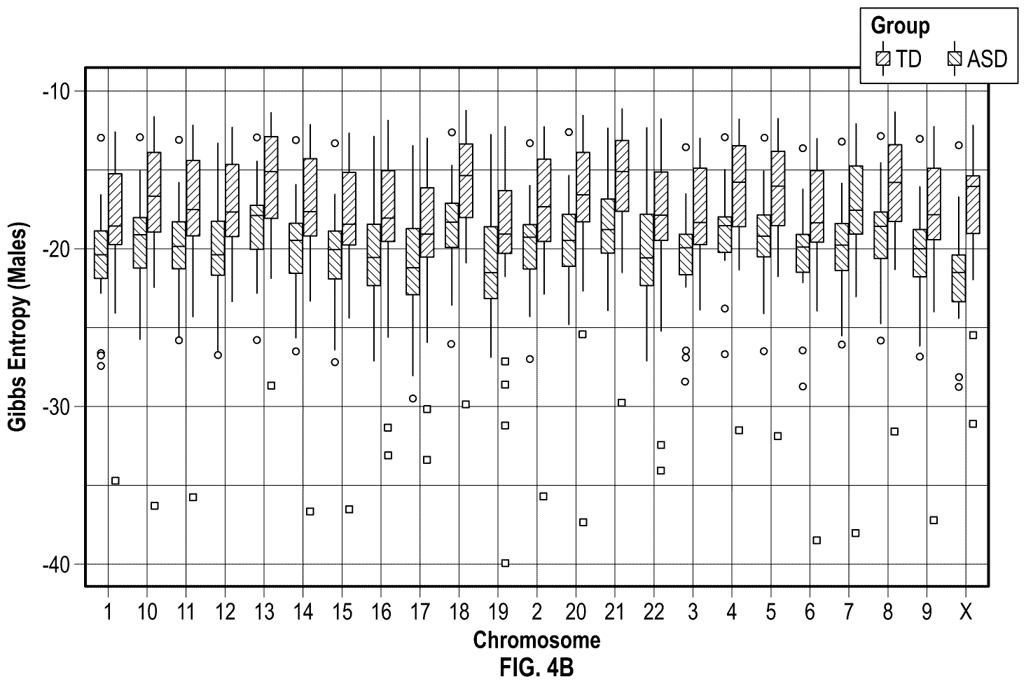

Results for the estimation of Gibbs entropy for every chromosome from controls and patients with autism are shown in the boxplots from FIGS. 4A-4B. For both sets, males and females, statistically significant differences were found between TD and ASD groups, in every chromosome. However, the boxplots also indicate the presence of atypical individuals which, in turn, suggests the existence of a structured population, where ASD individuals would experience the disorder at different severity levels.

Since the entropy variation ΔS=S_{ASD}−S_{TD} provides an estimation on the average of gain or loss of information (I=−ΔS), the boxplots also indicate a statistically significant loss of information (I<0) (on average) in the ASD group (higher entropy values) with respect to TD group (lower entropy values). Basically, ASD tissue cells experienced a loss of information translated into a loss of methylation regulatory signal typically found in healthy individuals.

Estimations of the discriminatory power of genes from identified essential enriched gene networks (as described in authors previous works) together with the entropy estimations can provide an assessment on the diseases severity level at a given stage. For example, from two patients carrying similar affected (essential) enriched gene networks, the one with lower Gibbs entropy experienced a lesser loss of information and, consequently, we would expect lower disease severity in such an individual.

Notice that, depending on the individual and on the disease, a loss of information would be associated with a negative or a positive effect on the individual's health condition. The loss of information observed in patients with cancer or with autism suggests a negative effect (in general) on patient conditions. Nevertheless, some children with ASD could carry methylation reprograming on gene networks associated with intellectual performance in mathematics, equipping the patient with good mathematical skills, as suggested in the network analysis of ASD group (data not shown).

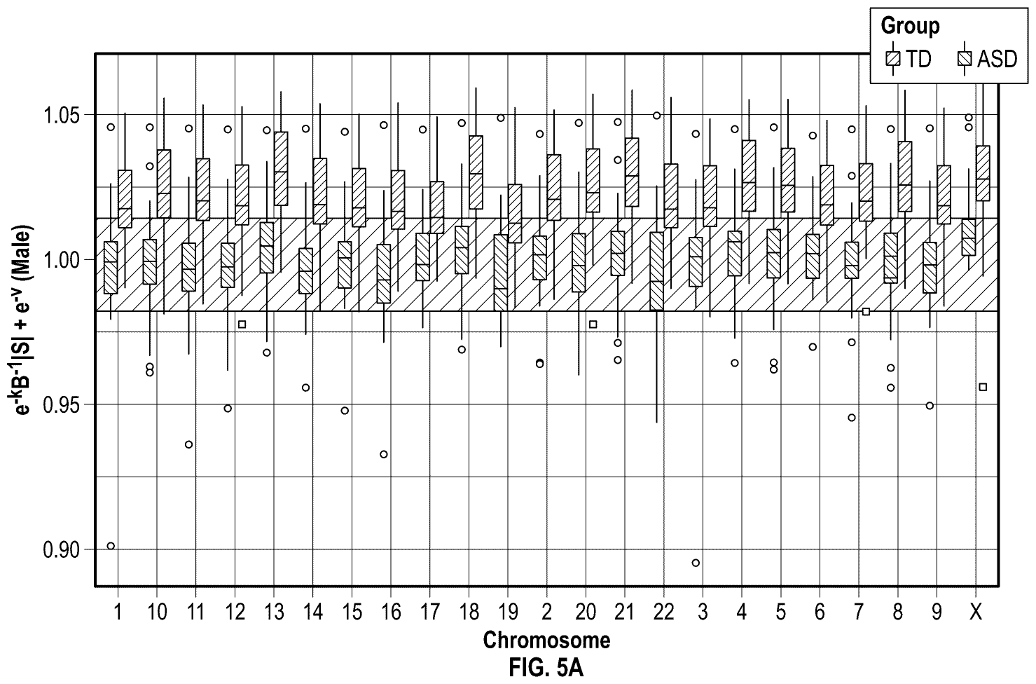

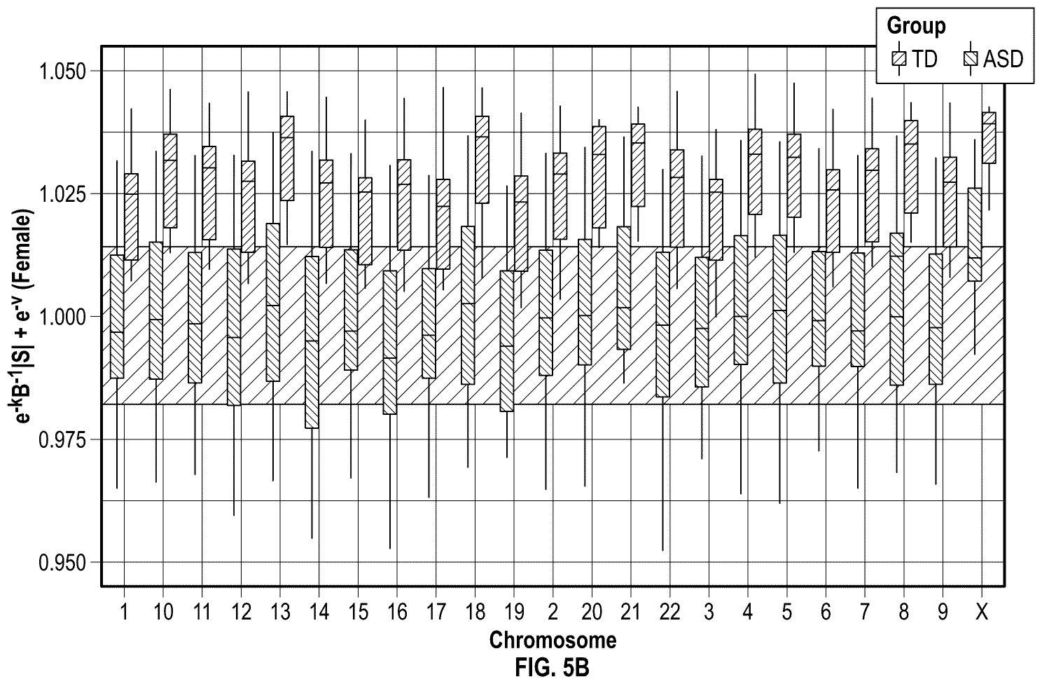

FIGS. 5A-5B show the analysis of random fluctuations in TD and ASD children. As shown in FIGS. 5A-5B, it must be expected that (depending on the tissue) the feedback control from the methylation regulatory system should keep the range of entropy fluctuations induced by exogenous forces tight to one. As shown in FIGS. 5A-5B, highly statistically significant differences were found between the entropy fluctuations from TD and ASD groups. However, the presence of outliers in TD group suggests a plausible effect of clinical conditions not detected in the evaluation tests applied for the identification of ASD, years later after the children were born. In other words, since the entropy fluctuation analysis is not restricted to any particular diseases, the observed outlier in TD (“healthy”) group would be the signal from an unknown clinical condition under developing in those individuals carrying extreme entropy fluctuations.

### Discussion

A theoretical premise to account for DNA methylation variation behavior is therefore presented. Results shown describe the information thermodynamics of cytosine methylation, extending well beyond the simple application of the probability density function for the information divergence as null hypothesis required for methylation analysis. Results confirm that members of the generalized gamma probability distribution family, as given by the generalized gamma probability density function, quantitatively summarize the statistical physics underlying spontaneous methylation variation driven by random fluctuations. Parameters from the generalized gamma probability density function carry information about channel capacity of molecular machines, relating to Shannon's capacity theorem.

In the context of Shannon's communication theory, the probability density function for the information divergence can be interpreted as a conditional probability density distribution. The conditional probability interpretation of methylation given by the conditional probability P_{y}(x) assumes that the message remains constant in the control population and that, under conditions of environmental variation or disease, changes in the message occur in some subpopulation, represented in treatment or patient datasets.

Consistent with Shannon's theory, with best encoding, the conditional probability density P_{y}(χ) indicates that if the recovered message at the receiving point is y, then P_{y}(χ) will decline exponentially with information divergence χ(x, y) between y and the message x produced by the source. If DNA methylation conforms to a communication system, then optimal coding of the methylation message is described in the probability density function for the information divergence as a logical application of Shannon's results.

Methylation changes that serve to support DNA thermal stability are expected to be present in highest frequency and with relatively small divergence values (left side of the probability density function in FIG. 1B). Observed data from control populations show information divergence values χ(x, y): χ(x, y)<χ_{α=0.05}≤X_{cutoff} to be small, representing background “noise” in the system. As shown in FIG. 1B, most of those values χ(x, y)>0 also hold the inequality χ(x, y)≤χ_{cutoff}, where χ_{cutoff} is some estimated value addressed to minimize the false positive rate in assessing differentially methylated positions (FIG. 1B, right side of the curve), representing treatment-associated variation. In practice, machine learning approaches can be applied to this estimation.

The methylation message is encoded within the mechanical properties of a DNA molecule. For example, flexibility or rigidity of the DNA double helix is required for regulating nucleosome folding and transcription factor (TF) binding to DNA sequence motifs. Depending on the DNA sequence context, the addition or removal of methyl groups to cytosine nucleotides is predicted to alter these local physical properties.

Gibbs entropy and Helmholtz free energy, as given by S=S_{classic}+S_{machine} and ΔF=−βS_{classic}−βS_{machine}=ΔF_{classic}+ΔF_{machine}, suggest a substantial distinction between classical statistical mechanics and statistical biophysics of the methylation process. The entropy contribution from molecular machines (enzymes) through conformational changes is included in function ϕ(α, δ). Application of these equations to experimental datasets can provide important biological insights. Results shown in Table 1 indicate that, as a thermodynamic state variable, entropy derived by S=S_{classic}+S_{machine} estimates the state of the methylation system consistent with phenotypic observations. Epigenetic memory lines in Arabidopsis produced an incremental effect on information loss observed in nm1 to mm3. In contrast, the met1 mutant, which undergoes genome-wide loss in CG methylation, provides a reference for extreme methylation change and information loss (Table 1).

Results presented in Table 2 show that Gibbs entropy estimated in methylome data from patients with different cancer types approaches values even higher than those in embryonic stem cells. DNA methylation repatterning appears to convey an associated increase in system entropy, placing cancer cells closer to undifferentiated somatic cells. Transformation of normal to cancer cells leads to an increase in entropy and consequent loss of information S=S_{normal cells}−S_{cancer cells}. Biological evidence similarly suggests that a loss of information from the original tissue occurs when cancer stem cells, a sub-population from within the tumor mass, derive from cancer cells. Results from Tables 1 and 2 agree with these known effects and the observations from this study suggest that transformation of healthy to cancer cells disrupts natural methylation programming and creates a genome-wide disordered state.

The experimental validation of each of the equations of this section in methylome datasets from human and Arabidopsis chromosomes is important to understanding the DNA methylation process. These equations indicate that fluctuation of methylation satisfies specific restrictions expressed in the quantitative relationships between magnitude of entropy fluctuations and expected value of J-information-divergence. The mean v and the variance σ² of J-information-divergence are integrally determined by parameter values from the conditional probability density Π_{y}(χ), consistent with Shannon's theory. Therefore, fluctuation constraints revealed by the equations of this section are concerned with preserving, with best encoding, fidelity of the methylation message at receiver point. This outcome presumably permits sufficient variation of the methylation signal to ensure organismal adaptation to environmental changes.

Samples reflecting extreme scenarios in Arabidopsis (met1) and human (breast cancer, metastasis and stem cells) did not adhere to the models set forth by the equations of this section (FIGS. 2 and 3). The met1 mutation leads to a nearly complete loss of CG gene-body methylation and substantial ectopic CHG and CHH genic and transposable element hypermethylation. Similarly, methylation reprogramming in cancer cells leads to massive loss of information as indicated by results shown in Table 2. However, the case of embryonic stem cells appears to be quite different from met1 and cancer cells, since DNA methylation is not necessarily required in this cellular context. Even with the complete loss of CG methylation by combined disruption of the three DNA methyltransferases Dnmt1, Dnmt3a, and Dnmt3b, there is minimal change in cellular phenotype. As pluripotent undifferentiated cells, there may be no need to preserve stem cells in a specific epigenomic state prior to differentiation to specialized tissues.

While the primary goal of this study was to establish a theoretical basis for understanding DNA methylation behavior, our results suggest that practical implications of entropy estimates may prove significant for early diagnostics and assessing change in epigenetic states. These data, in both plant and human disease, indicate that detection of transitions in epigenomic states that occur, for example, during plant response to environmental change or human pre-cancerous cell transitions, may be feasible on the basis of the type of physical-informational chromosome data presented.

### EXAMPLES

### Biological Experimental Datasets

The Arabidopsis thaliana methylome datasets (reported in Table 1) derive from whole-genome bisulfite sequencing of samples from msh1 memory (generations 1-6) and non-memory (generation 1) sibling plants (5 plants/generation) with isogenic Col-0 wild-type control (5 plants). Datasets were downloaded from the Gene Expression Omnibus (GEO) Series GSE129303a and GSE118874.

The methylome datasets for met1 mutant and corresponding wildtype (3 samples each) were taken from the GEO Series GSE122394. The fastq files from Arabidopsis methylome met1 mutant and corresponding wildtype datasets were downloaded from the European Nucleotide Archive (ENA, https://www.ebi.ac.uk/ena/browser/home). Raw read counts for met1 methylated and non-methylated cytosines for further methylation analysis were obtained as follows: Raw sequencing reads were quality-controlled with FastQC (version 0.11.5), trimmed with TrimGalore! (version 0.4.1) and Cutadapt (version 1.15), then aligned to the TAIR10 reference genome using Bismark (version 0.19.0) with bowtie2 (version 2.3.3.1). The deduplicate_bismark function in Bismark with default parameters was used to remove duplicated reads and reads with coverage greater than 500 were removed to control PCR bias. Methylated Cs (COV files) were acquired from Bismark methylation extractor with default parameters.

Human cancer and healthy tissue control datasets (Table 2) were downloaded from the GEO Series GSE52271. Blood B-cells CD19 (GSM1279518) was used as reference in the computation of information divergences J-divergences (JD). The Bi-seq dataset of Naïve Human Pluripotent Cells have GEO accessions: GSM2041690, GSM2041691, and GSM2041692. A more detailed description of these datasets is given below.

### Material and Methods Used to Compute Results Presented in Table 1

### 1. Hellinger and J Information Divergences of Methylation Levels

The methylation level pᵢⱼ for an individual i cytosine site j corresponds to a probability vector p^{ij}=(pᵢⱼ, 1−pᵢⱼ). Then, the information divergence between methylation levels. The methylation level p¹j and p²j from individuals 1 and 2 at site j is the divergence between the vectors p¹j=(p₁j, 1−p₁j) and p²j=(p₂ⱼ, 1−p₂ⱼ). If the vector of coverage is supplied, then the information divergence is estimated according to the formula:

$$H D = \frac{2 ( {n}_{1} + 1 ) ( {n}_{2} + 1 )}{{n}_{1} + {n}_{2} + 2} ( {( \sqrt{( {p}_{1 j} )} - \sqrt{( {p}_{2 j} )} )}^{2} + {( \sqrt{( 1 - {p}_{1 j} )} - \sqrt{( 1 - {p}_{2 j} )} )}^{2} )$$

This formula corresponds to Hellinger divergence as given by the inventors of the present disclosure in the first formula from Theorem 1 from Kundariya, H., et al., “MSH1-induced heritable enhanced growth vigor through grafting is associated with the RdDM pathway in plants.” Nat Commun 11, 5343 (2020), hereby incorporated by reference in its entirety herein. Otherwise:

$$H D = {( \sqrt{( {p}_{1 j} )} - \sqrt{( {p}_{2 j} )} )}^{2} + {( \sqrt{( 1 - {p}_{1 j} )} - \sqrt{( 1 - {p}_{2 j} )} )}^{2}$$

It is important to observe that several filtering conditions are provided to select biological meaningful cytosine positions, which prevent to carry experimental errors in the downstream analyses. By filtering the read count bad quality data can be removed, which would be in the edge of the experimental error originated by the BS-seq sequencing. The user can check whether cytosine positions used in the analysis are biological meaningful. For example, a cytosine position with counts mC1=10 and uC1=20 in the ‘ref’ sample and mC2=1 and uC2=0 in an ‘indv’ sample will lead to methylation levels p1=0.333 and p2=1, respectively, and TV=p2−p1=0.667, which apparently indicates a hypermethylated site. However, there are not enough reads supporting p2=1. A Bayesian estimation of TV will reveal that this site would be, in fact, hypomethylated. So, the best practice will be the removing of sites like that. This particular case is removed under the default settings: min.coverage=4,min.meth=4, and min.umeth=0 (see example for function uniqueGRfilterByCov, called by ‘estimateDivergence’, described in the subsequent section: infra).

### 2. The J-Divergence

Given the methylation levels from two individuals at a given cytosine site, this function computes the J-information divergence (JD) between methylation levels. The motivation to introduce JD in Methyl-IT is founded on the enumerated items below:
1. It is a symmetrized form of Kullback-Leibler divergence: D_{KL}(P∥Q), Kullback and Leibler themselves actually defined the divergence as:

$$J D = \frac{1}{2} ( {D}_{KL} ( P ‖ Q ) + {D}_{KL} ( Q ‖ P ) )$$

which is symmetric and nonnegative, where the probability distributions P and Q are defined on the same probability space.
2. In general, JD is highly correlated with Hellinger divergence, which is the main divergence currently used in Methyl-IT (see the following example for function estimateDivergence:

```
estimateDivergence(
    ref,
    indiv,
    Bayesian = FALSE,
    init.pars = NULL,
    via.optim = TRUE,
    columns = c(mC = 1, uC = 2),
    min.coverage = 4,
    and.min.cov = TRUE,
    min.meth = 4,
    min.umeth = 0,
    min.sitecov = 4,
    high.coverage = NULL,
    percentile = 0.9999,
    JD = FALSE,
    jd.stat = FALSE,
    ignore.strand = FALSE,
    y.centroid = NULL,
    num.cores = multicoreWorkers( ),
    tasks = 0L,
    meth.level = FALSE,
    loss.fun = c(“linear”, “huber”, “smooth”, “cauchy”,
      “arctg”),
    logbase = 2,
    verbose = TRUE,
  ...
)
```

The estimateDivergence function prepares the data for the estimation of information divergences and works as a wrapper calling the functions that compute selected information divergences of methylation levels. In the downstream analysis, the probability distribution of a given information divergence is used in Methyl-IT as the null hypothesis of the noise distribution, which permits, in a further signal detection step, the discrimination of the methylation regulatory signal from the background noise. For the current version, two information divergences of methylation levels are computed by default: 1) Hellinger divergence (H) and 2) the total variation distance (TVD). In the context of methylation analysis TVD corresponds to the absolute difference of methylation levels. Here, although the variable reported is the total variation (TV), the variable actually used for the downstream analysis is TVD. Once a differentially methylated position (DMP) is identified in the downstream analysis, TV is the standard indicator of whether the cytosine position is hyper- or hypo-methylated. The option to compute the J-information divergence (JD) is currently provided. The motivation to introduce this divergence is given in the help of function estimateJDiv.
3. By construction, the unit of measurement of JD is given in units of bit of information, which set the basis for further information-thermodynamics analyses.

Methylation levels pᵢⱼ at a given cytosine site i from an individual j, lead to the probability vectors p^{ij}=(pᵢⱼ, 1−pᵢⱼ). Then, the J-information divergence between the methylation levels p^{ij} and qⁱ=(qᵢ, 1−qᵢ) (used as reference individual), is given by the expression:

$$J D ( {p}^{ij} , {q}^{i} ) = \frac{1}{2} ( ( {p}_{ij} - {q}_{i} ) log ( \frac{{p}_{ij}}{{q}_{i}} ) + ( {q}_{i} - {p}_{ij} ) log ( \frac{( 1 - {p}_{ij} )}{( 1 - {q}_{i} )} ) ) .$$

The statistic with asymptotic Chi-squared (χ²) distribution is based on the statistic for D_{KL}. That is:

$$2 \frac{( {n}_{1} + 1 ) ( {n}_{2} + 1 )}{( {n}_{1} + {n}_{2} + 2 )} J D ( {p}^{ij} , {q}^{i} )$$

where n₁ and n₂ are the total counts (coverage in the case of methylation) used to compute the probabilities p^{ij} and qⁱ. A basic Bayesian correction is added to prevent zero counts.

### 3. Methylome Data—Human Dataset

All the methylome data sets were taken from Gene Expression Omnibus (GEO) data base.
b) Bi-seq data sets from Naïve Human Pluripotent Cells has GEO accession: GSM2041690, GSM2041691, and GSM2041692.
c) Human cancer types and corresponding normal tissue with GEO accessions: GSE52271 and GSE56763.
   1. Brain white matter (GSM1279516)
   2. Brain Glioma (GSM1279532)
   3. Breast normal (GSM1279517)
   4. Breast cancer (GSM1279514)
   5. Breast metastasis (GSM1279513)
   6. Colon normal (GSM1279519)
   7. Colon cancer (GSM1279521)
   8. Colon metastasis (GSM1279520)
   9. Lung normal (GSM1279527)
   10. Lung cancer (GSM1279524)
   11. Blood B-cells CD19, normal (GSM1279518)

The Blood B-cells CD19 sample was used as reference in the computation of information divergences: Hellinger (HD) and J-divergences (JD).

Public raw data sets of methylation profiling by high throughput sequencing (with accession number GSE178203) from patients with autism spectrum disorder (ASD) and control (typical development, TD) were downloaded from Gene Expression Omnibus (GEO) and reanalyzed with MethylIT. The data set consists of placenta tissue from 42 male (20 TD and 22 ASD) and 23 female individuals (10 TD and 12 ASD). The raw data was originally reported in a published study from reference.

Specifically, placenta tissue from patients with autism spectrum disorder (ASD) and control (typical development, TD) typical development with GEO accession GSE178203.
a) TD male children
   1. GSM5381715
   2. GSM5381716
   3. GSM5381720
   4. GSM5381726
   5. GSM5381728
   6. GSM5381730
   7. GSM5381733
   8. GSM5381738
   9. GSM5381741
   10. GSM5381745
   11. GSM5381750
   12. GSM5381751
   13. GSM5381753
   14. GSM5381754
   15. GSM5381755
   16. GSM5381759
   17. GSM5381760
   18. GSM5381762
   19. GSM5381772
   20. GSM5381774
b) ASD male children
   1. GSM5381713
   2. GSM5381718
   3. GSM5381722
   4. GSM5381724
   5. GSM5381732
   6. GSM5381737
   7. GSM5381739
   8. GSM5381740
   9. GSM5381743
   10. GSM5381744
   11. GSM5381746
   12. GSM5381747
   13. GSM5381748
   14. GSM5381749
   15. GSM5381752
   16. GSM5381756
   17. GSM5381757
   18. GSM5381758
   19. GSM5381761
   20. GSM5381764
   21. GSM5381767
   22. GSM5381768
c) Female TD children
   1. GSM5381710
   2. GSM5381711
   3. GSM5381714
   4. GSM5381719
   5. GSM5381723
   6. GSM5381727
   7. GSM5381731
   8. GSM5381734
   9. GSM5381765
   10. GSM5381766
d) ASD female children
   1. GSM5381712
   2. GSM5381717
   3. GSM5381721
   4. GSM5381725
   5. GSM5381729
   6. GSM5381735
   7. GSM5381736
   8. GSM5381742
   9. GSM5381763
   10. GSM5381769
   11. GSM5381770
   12. GSM5381771
   13. GSM5381773
      4. Methylome Data—Arabidopsis thaliana Datatset

The Arabidopsis thaliana methylome datasets of msh1 memory and non-memory (normal looking) sibling plants were derived from the msh1 mutant. Basically, as described in reference (35) (main text), a transgene positive plant was self-pollinated and transgene was segregated in subsequent generation. Of transgene null plants, 20% plants displayed delayed in flowering, smaller in size, and lighter green termed as memory phenotype. Memory plants were self-pollinated for six generations and plants from each generation were Bisulfite sequenced. The dataset generation 2-6 can be accessed with GEO accession number GSE129303 and GSE118874.

The RData files with J-information-divergence and with the best fitted models and corresponding goodness-of-fit used in all the computation reported in the main text are computed with the J-divergence function as outlined above.

### 5. Methylation Analysis

Methylation analysis was accomplished using aspects of the MethylT R package (0.3.2.2) that was described by the present inventors in technical literature that was incorporated by reference supra, including but not limited to, U.S. provisional patent application Ser. No. 63/323,690, Sanchez et al., “Discrimination of DNA Methylation Signal from Background Variation for Clinical Diagnostics”, Int. J. Mol. Sci. 20, 5343 (2019), Sanchez et al., “Information thermodynamics of cytosine DNA methylation.” PLOS One 11, e0150427 (2016), and Sanchez et al., “Genome-wide discriminatory information patterns of cytosine DNA methylation”, Int. J. Mol. Sci. 17, 938 (2016). New R scripts with the methylation analysis are available below, in the subsections that begin with “R Script”, infra.

TABLE 4
Best fitted PDF model parameters for GG distribution used
to estimate Gibb entropy and Helmholtz free energy.

| | Cell | | | | | |
|---|---|---|---|---|---|---|
| | Chromosome 7 | | | Chromosome 9 | | |
| Tissue | α | β | δ | α | β | δ |
| hesc_1 | 32.18209 | 1.25683 | 0.02908 | 30.86183 | 1.26053 | 0.02962 |
| hesc_2 | 32.95730 | 1.21850 | 0.02652 | 30.70496 | 1.22164 | 0.02787 |
| hesc_3 | 32.32213 | 1.22118 | 0.02841 | 30.82312 | 1.22469 | 0.02911 |
| Brain | 0.61745 | 0.04658 | 0.98026 | 0.58150 | 0.04186 | 1.05729 |
| Glioma | 1.01469 | 1.86909 | 0.33113 | 0.80394 | 1.21351 | 0.44878 |
| Breast | 0.49217 | 0.03517 | 1.20660 | 0.49124 | 0.03777 | 1.19956 |
| Breast Cancer | 0.61368 | 1.57812 | 0.56625 | 0.30328 | 0.02639 | 1.86484 |
| Breast Metastasis | 51.19292 | 5.12655 | 0.00604 | 46.24423 | 4.64894 | 0.00645 |
| Colon | 0.56040 | 0.05445 | 1.00374 | 0.55697 | 0.05657 | 1.00844 |
| Colon Cancer | 0.52759 | 0.09799 | 0.99489 | 0.49492 | 0.07397 | 1.09942 |
| Colon Metastasis | 0.57161 | 0.31631 | 0.76750 | 0.50731 | 0.17379 | 0.94482 |
| Lung | 0.70752 | 0.06819 | 0.78636 | 0.68826 | 0.06580 | 0.81404 |
| Lung Cancer | 0.29639 | 0.00827 | 2.01754 | 0.32413 | 0.01371 | 1.82223 |
| | Chromosome 17 | | | Chromosome 22 | | |
| hesc_1 | 30.64292 | 1.26484 | 0.03295 | 25.45504 | 1.27350 | 0.03822 |
| hesc_2 | 30.32504 | 1.22553 | 0.03123 | 24.31235 | 1.23421 | 0.03754 |
| hesc_3 | 30.37249 | 1.22872 | 0.03266 | 25.24652 | 1.23734 | 0.03786 |
| Brain | 0.56368 | 0.03757 | 1.12392 | 0.53325 | 0.03330 | 1.20079 |
| Glioma | 0.46217 | 0.35588 | 0.88572 | 1.54098 | 2.71554 | 0.19747 |
| Breast | 0.42953 | 0.02284 | 1.43882 | 0.42680 | 0.02504 | 1.43110 |
| Breast Cancer | 0.17236 | 0.00001 | 5.42893 | 0.26865 | 0.00941 | 2.22685 |
| Breast Metastasis | 1.14748 | 3.89031 | 0.26122 | 2.21365 | 5.01926 | 0.13086 |
| Colon | 0.52358 | 0.04668 | 1.08923 | 0.53935 | 0.05484 | 1.04262 |
| Colon Cancer | 0.43052 | 0.03723 | 1.36760 | 0.48313 | 0.07303 | 1.11830 |
| Colon Metastasis | 0.41534 | 0.05582 | 1.33030 | 0.48874 | 0.11388 | 1.05197 |
| Lung | 0.67463 | 0.06668 | 0.84053 | 0.66433 | 0.06827 | 0.85011 |
| Lung Cancer | 0.32601 | 0.01972 | 1.68885 | 0.34469 | 0.01545 | 1.73365 |

### Computational Tools and Statistical Analysis

The estimations of J-divergences, best nonlinear fitted model to member of the generalized gamma distribution (the probability density function for the information divergence and the more general distribution including the location parameter μ), Gibbs entropy, and Helmholtz free energy were accomplished using MethylIT functions gibb_entropy and helmholtz_free_energy, respectively. The estimations of the Boltzmann's factors shown in FIG. 2 were accomplished using MethylIT function boltzman_factor. All R scripts for Tables 1-3 results are available in the sections, supra, titled: R Script for the Analysis of cancer data set, R Script for the Analysis of Arabidopsis data set, and R Script for the Analysis of Entropy fluctuations.

The group comparison shown in Table 1 was accomplished in the lme4 R package (version 1.1-27.1) applying a linear mixed model with chromosome random effects with formula:

$$entropy = group + ( 1 \vert chromosome ) .$$

TABLE 5

| Entropy | C1 | C2 | C3 | T1 | T2 | T3 |
|---|---|---|---|---|---|---|
| Theoretical Equation | 3.941 | 3.918 | 3.929 | 4.457 | 4.469 | 4.515 |
| Full numerical estimation | 4.061 | 4.043 | 4.047 | 4.571 | 4.580 | 4.630 |

The differences between the results of the theoretical equation with the results of the full numerical estimation are between the limits of the experimental error. That is, if someone applies some arbitrary theoretical information divergence to the methylation levels and computes a full numerical estimation of the Gibb or Boltzmann entropy, then such a person/company will get results that will emulate our entropy results, up the limit of a constant value.

### R Script for the Analysis of Cancer Data Set

This data set was downloaded from the Gene Expression Omnibus (GEO) to a local folder and read into R.
library(MethylIT)

### 1. Reading Data Sets

```
folder <− “~/Cancer/GSE52271/”
files = list.files(path = folder, pattern = “txt.gz”)
files = paste0(folder, files)
folder <− “/data/HumanMethy/Cancer/GSE56763/”
files = c(files, paste0(folder, list.files(path = folder,
  pattern = “txt.gz”)))
# [1] “~/Cancer/GSE52271/GSM1279516_CpGcontext.Brain_W.txt.gz”
# [2] “~/Cancer/GSE52271/GSM1279517_CpGcontext.Breast.txt.gz”
# [3] “~/Cancer/GSE52271/GSM1279518_CpGcontext.CD19.txt.gz”
# [4] “~/Cancer/GSE52271/GSM1279519_CpGcontext.Colon.txt.gz”
# [5] “~/Cancer/GSE52271/GSM1279520_CpGcontext.Colon_M.txt.gz”
# [6] “~/Cancer/GSE52271/GSM1279521_CpGcontext.Colon_P.txt.gz”
# [7] “~/Cancer/GSE52271/GSM1279522_CpGcontext.H1437.txt.gz”
# [8] “~/Cancer/GSE52271/GSM1279523_CpGcontext.H157.txt.gz”
# [9] “~/Cancer/GSE52271/GSM1279524_CpGcontext.H1672.txt.gz”
# [10] “~/Cancer/GSE52271/GSM1279527_CpGcontext.Lung.txt.gz”
# [11] “~/Cancer/GSE52271/GSM1279532_CpGcontext.U87MG.txt.gz”
# [12] “~/Cancer/GSE56763/GSM1279513_CpGcontext.468LN.txt.gz”
# [13] “~/Cancer/GSE56763/GSM1279514_CpGcontext.468PT.txt.gz”
sn = c(“Brain”, “Breast”, “CD19”, “Colon”, “Colon.M”,
  “ColonCancer”, “Adenocarcinoma”, “SquamousCancer”,
  “LungCancer”, “Lung”, “Glioma”, “BreastMeta”,
  “BreastCancer”)
LR. = readCounts2GRangesList
  (filenames = files, sample.id = sn,
  columns = c(seqnames = 1, start = 2, mC = 3, uC = 4 ),
  pattern = “\hat{ }[\hat{ }MY]”)
LR <− c(LR, LR.)
names(LR[−6])
save(LR, file = “~/Cancer/RData/LR_cancer.R”)
```

### 2. Hellinger Divergence

### Estimation of Hellinger Divergence

```
load(“~/Cancer/RData/LR_cancer.R”)
HD = estimateDivergence(ref = LR$CD19,
  indiv = LR[−6],
  Bayesian = TRUE,
  min.coverage = 8,
  high.coverage = 500,
  min.meth = 3,
  min.umeth = 0,
  percentile = 0.999,
  JD = TRUE,
  num.cores = 60L,
  verbose = FALSE)
save(HD, file = “~/Cancer/RData/hd_cancer.RData”)
```

For the sake of reducing computational work, only J-divergences from chromosomes 7, 9, 17, and 22 are of interest.

```
chrs <− c(“7”, “9”, “17”, “22”)
nams <− names(HD)
# [1] “hesc_1” “hesc_2” “hesc_3” “Brain” “Breast”
# [6] “Colon” “Colon.M” “ColonCancer” “Adenocarcinoma”
  “SquamousCancer”
# [11] “LungCancer” “Lung” “Glioma” “BreastMeta”
  “BreastCancer”
jd_chr <− function(jd, chr) lapply(jd, function(x) {
  seqlevels(x, pruning.mode = “coarse”) <− chr
  x <− x[, 10]
  return(x)
  }, keep.attr = TRUE
)
jd <− vector(“list”, 4)
for (k in seq_along(chrs)) {
  cat(“\n *** ========== Processing chromosome: ”, chrs[k],
    “ ...\n”)
  jd[[ k ]] <− jd_chr(jd = HD, chr = chrs[ k ])
}
names(jd) <− paste0(“chr”, chrs)
save(jd, file = “~/Cancer/RData/jd_cancer_datasets.RData”,
  compress = “xz”)
```

### 3. Nonlinear Regression for JD. Only GGamma

The last file can be download from GitLab using the following script:

```
## ===== Download methylation data from GitLab ========
url <− paste0(“https://git.psu.edu/genomath/datasets/-/raw/”,
  “main/cancer_data/jd_cancer_datasets.RData”)
temp <− tempfile(fileext = “.RData”)
download.file(url = url, destfile = temp)
load(temp)
file.remove(temp); rm(temp, url)
chrs <− c(“7”, “9”, “17”, “22”)
gof <− vector(“list”, 4)
for (k in seq_along(chrs)) {
  cat(“\n *** ========== Processing chromosome: ”, k, “
    ...\n”)
  gof[[ k ]] <− gofReport(HD = jd,
    model = “GGamma3P”,
    column = 1,
    num.cores = 40L,
    verbose = TRUE
  )
}
names(gof) <− paste0(“chr”, chrs)
```

The results from the nonlinear fit can be downloaded from the GitLab

```
## ===== Download methylation data from GitLab ========
url <− paste0(“https://git.psu.edu/genomath/datasets/-/raw/”,
  “main/cancer_data/jdiv_gof_per-
  chr_gwf_ggamma3p_cancer.RData”)
temp <− tempfile(fileext = “.RData”)
download.file(url = url, destfile = temp)
load(temp)
file.remove(temp); rm(temp, url)
#> [1] TRUE
gof
#> $chr7
```

| #> | alpha | scale | psi |
|---|---|---|---|
| #> hesc_1 | 32.1820949 | 1.256833168 | 0.029080812 |
| #> hesc_2 | 32.9572994 | 1.218499579 | 0.026518849 |
| #> hesc_3 | 32.3221334 | 1.221178069 | 0.028409356 |
| #> Brain | 0.6114547 | 0.044578283 | 1.000000000 |
| #> Glioma | 1.0000000 | 0.337197037 | 1.846401452 |
| #> Breast | 0.4921686 | 0.035167582 | 1.206604336 |
| #> BreastCancer | 0.4184692 | 0.457555710 | 1.000000000 |
| #> BreastMeta | 51.1929207 | 5.126553336 | 0.006039215 |
| #> Colon | 0.5630709 | 0.054784402 | 1.000000000 |
| #> ColonCancer | 0.5257514 | 0.096760982 | 1.000000000 |
| #> Colon.M | 0.4805484 | 0.176995395 | 1.000000000 |
| #> Lung | 0.7075234 | 0.068188212 | 0.786364123 |
| #> LungCancer | 0.2963886 | 0.008268955 | 2.017535427 |
| #> Adenocarcinoma | 48.5899874 | 3.715311025 | 0.006119806 |
| #> SquamousCancer | 60.3389779 | 4.253576158 | 0.005729587 |

#>
#> $chr9
#>

| | | | |
|---|---|---|---|
| #> | alpha | scale | psi |
| #> hesc_1 | 30.8618318 | 1.26053254 | 0.029619450 |
| #> hesc_2 | 30.7049640 | 1.22164475 | 0.027868363 |
| #> hesc_3 | 30.8231174 | 1.22469394 | 0.029114706 |
| #> Brain | 0.6043353 | 0.04661833 | 1.000000000 |
| #> Glioma | 0.8039367 | 1.21350590 | 0.448777331 |
| #> Breast | 0.4912445 | 0.03777369 | 1.199561523 |
| #> BreastCancer | 0.3032793 | 0.02638795 | 1.864844905 |
| #> BreastMeta | 46.2442290 | 4.64894361 | 0.006453787 |
| #> Colon | 0.5611143 | 0.05750991 | 1.000000000 |
| #> ColonCancer | 0.4949193 | 0.07397311 | 1.099419584 |
| #> Colon.M | 0.4889919 | 0.15152698 | 1.000000000 |
| #> Lung | 0.6882550 | 0.06580041 | 0.814037167 |
| #> LungCancer | 0.3241347 | 0.01371265 | 1.822229516 |
| #> Adenocarcinoma | 3.8047732 | 3.53206742 | 0.077239217 |
| #> SquamousCancer | 53.8674361 | 4.37722477 | 0.005879299 |

#>
#> $chr17
#>

| | | | |
|---|---|---|---|
| #> | alpha | scale | psi |
| #> hesc_1 | 30.6429199 | 1.26484391 | 0.03294777 |
| #> hesc_2 | 30.3250420 | 1.22553227 | 0.03122785 |
| #> hesc_3 | 30.3724908 | 1.22872417 | 0.03266358 |
| #> Brain | 0.6095099 | 0.04760985 | 1.00000000 |
| #> Glioma | 0.4250431 | 0.25807283 | 1.00000000 |
| #> Breast | 0.4295276 | 0.02283972 | 1.43881864 |
| #> BreastCancer | 0.4445777 | 0.20589533 | 1.00000000 |
| #> BreastMeta | 1.0000000 | 0.30804779 | 3.55751532 |
| #> Colon | 0.5235813 | 0.04667787 | 1.08923338 |
| #> ColonCancer | 0.4305240 | 0.03722721 | 1.36759912 |
| #> Colon.M | 0.4153388 | 0.05582166 | 1.33029818 |
| #> Lung | 0.6746254 | 0.06667974 | 0.84052911 |
| #> LungCancer | 0.3260148 | 0.01972477 | 1.68885054 |
| #> Adenocarcinoma | 0.4221597 | 0.27942832 | 1.00000000 |
| #> SquamousCancer | 3.0500842 | 4.14873039 | 0.08800507 |

#>
#> $chr22
#>

| | | | |
|---|---|---|---|
| #> | alpha | scale | psi |
| #> hesc_1 | 25.4550427 | 1.273497760 | 0.038224963 |
| #> hesc_2 | 24.3123549 | 1.234211929 | 0.037544184 |
| #> hesc_3 | 25.2465158 | 1.237336132 | 0.037857998 |
| #> Brain | 0.5332527 | 0.033300540 | 1.200789320 |
| #> Glioma | 1.5409763 | 2.715541968 | 0.197466075 |
| #> Breast | 0.4267980 | 0.025043442 | 1.431104067 |
| #> BreastCancer | 0.2686536 | 0.009410411 | 2.226846411 |
| #> BreastMeta | 2.2136545 | 5.019260707 | 0.130859588 |
| #> Colon | 0.5393467 | 0.054842595 | 1.042623372 |
| #> ColonCancer | 0.4831316 | 0.073028969 | 1.118302772 |
| #> Colon.M | 0.4887350 | 0.113881221 | 1.051972307 |
| #> Lung | 0.6643343 | 0.068268936 | 0.850107005 |
| #> LungCancer | 0.3446926 | 0.015454173 | 1.733652211 |
| #> Adenocarcinoma | 0.4233096 | 0.294426097 | 1.000000000 |
| #> SquamousCancer | 45.0958702 | 4.177438243 | 0.006215187 |

### 4. Gibb Entropy

sapply(gofs, gibb_entropy)

| #> | chr7 | chr9 | chr17 | chr22 |
|---|---|---|---|---|
| #> hesc_1 | 1.981123 | 1.9883104 | 2.077586 | 2.146658 |
| #> hesc_2 | 1.645168 | 1.6405274 | 1.787076 | 1.839961 |
| #> hesc_3 | 1.724381 | 1.7281328 | 1.833183 | 1.897203 |
| #> Brain | −16.507387 | −16.1304274 | −15.958911 | −15.402672 |
| #> Breast | −14.558528 | −14.0775605 | −13.606114 | −12.911931 |
| #> Colon | −14.782326 | −14.3794233 | −14.416785 | −13.965082 |
| #> Colon.M | −5.177822 | −6.4419314 | −7.776171 | −7.686259 |
| #> ColonCancer | −10.087572 | −10.3264955 | −10.711638 | −10.036097 |
| #> Adenocarcinoma | 1.303486 | 0.3017815 | −1.685461 | −1.242498 |
| #> SquamousCancer | 5.102169 | 3.8577939 | −0.415287 | 1.059315 |
| #> LungCancer | −8.026692 | −8.5201005 | −7.807797 | −9.854450 |
| #> Lung | −16.734519 | −16.5538102 | −15.969389 | −15.622140 |
| #> Glioma | 3.596377 | −2.0063424 | −2.325970 | −0.970184 |
| #> BreastMeta | 4.728624 | 3.2369647 | 6.447846 | 2.882476 |
| #> BreastCancer | 2.387586 | −1.2706309 | −4.081463 | −1.465153 |

The entropy units are: J×K⁻¹×mol⁻¹.

### 5. Helmholtz Free Energy

sapply(gofs, helmholtz_free_energy)

| #> | chr7 | chr9 | chr17 | chr22 |
|---|---|---|---|---|
| #> hesc_1 | −614.4454 | −616.67446 | −644.3634 | −665.7859 |
| #> hesc_2 | −510.2487 | −508.80957 | −554.2617 | −570.6638 |
| #> hesc_3 | −534.8169 | −535.98039 | −568.5616 | −588.4176 |
| #> Brain | 5119.7662 | 5002.85207 | 4949.6563 | 4777.1387 |
| #> Breast | 4515.3273 | 4366.15538 | 4219.9361 | 4004.6353 |
| #> Colon | 4584.7383 | 4459.77814 | 4471.3659 | 4331.2703 |
| #> Colon.M | 1605.9016 | 1997.96503 | 2411.7793 | 2383.8932 |
| #> ColonCancer | 3128.6605 | 3202.76259 | 3322.2146 | 3112.6954 |
| #> Adenocarcinoma | −404.2761 | −93.59753 | 522.7458 | 385.3606 |
| #> SquamousCancer | −1582.4378 | −1196.49477 | 128.8012 | −328.5467 |
| #> LungCancer | 2489.4784 | 2642.50917 | 2421.5882 | 3056.3578 |
| #> Lung | 5190.2109 | 5134.16423 | 4952.9060 | 4845.2066 |
| #> Glioma | −1115.4162 | 622.26709 | 721.3996 | 300.9026 |
| #> BreastMeta | −1466.5829 | −1003.94462 | −1999.7996 | −894.0000 |
| #> BreastCancer | −740.5097 | 394.08618 | 1265.8658 | 454.4172 |

The energy units are: J×mol⁻¹.

### R Script for the Analysis of Arabidopsis Data Set

The R script example given here is limited to the 3rd generation, but it can be extended to all generations.

library(MethylIT)
library(MethylIT)
library(ggplot2)
library(ggpmisc)
library(dplyr)

For the sake of brevity the analysis is applied here only to the wildtype 3rd generation control and to memory line 3rd generation. The same R script was applied to all the set of samples.

If read count datasets available at GEO database, then MethylIT function getGEOSuppFiles can be used to download read count datasets from GEO. Users can always download manually by themselves and then read them into R with function readCounts2GRangesList.

1. Download Files from GEO

### Wildtype Control Samples:

```
wt3_files = getGEOSuppFiles(GEO = c(“GSM3704281”,
    “GSM3704282”, “GSM3704283”, “GSM3704284”,
    “GSM3704285”),
  verbose = FALSE)
```

Memory line 3rd generation:

```
mm3_files = getGEOSuppFiles (GEO =
c(“GSM3704257”, “GSM3704258”,
    “GSM3704259”, “GSM3704260”, “GSM3704261”) ,
  verbose = FALSE)
```

### 2. Reading Datasets

### Reading Wildtype Control Samples:

```
sn = c(“wt3_1”, “wt3_2”, “wt3_3”, “wt3_4”, “wt3_5”)
wt3 = readCounts2GRangesList (filenames = wt3_files, sample.id
    = sn,
  columns = c(seqnames = 1, start = 2, mC = 4, uC = 5 ),
  verbose = FALSE)
```

### Reading Memory-Line 3rd Generation Dataset:

```
sn = c(“m3_1”, “m3_2”, “m3_3”, “m3_4”, “m3_5”)
mm3 = readCounts2GRangesList(filenames = mm3_files, sample.id
    = sn,
  columns = c(seqnames = 1, start = 2, mC = 4, uC = 5 ),
  verbose = FALSE)
```

### 3. The Reference Sample

```
ref <− poolFromGRlist(LR = wt3, stat = “sum”,
  columns = 1:2,
  num.cores = 5L,
  verbose = TRUE)
```

### 4. J-Divergence

```
ptm <− proc.time( )
idiv <− estimateDivergence(ref = ref,
  indiv = wt3,
  Bayesian = TRUE,
  JD = TRUE,
  min.coverage = 5,
  high.coverage = 500,
  percentile = 0.999,
  num.cores = 3L,
  verbose = FALSE)
cat((proc.time( ) − ptm) [3]/60, “minutes.”, date( )) # in min
```

### 5. Estimation of the Best Fitted Probability Distribution Model

Depending on your computation capability, this step takes considerable computational time. The RData files with information divergence values can be download from GitLab using the following script:

```
## ===== Download methylation data from GitLab ========
url <− paste0(“https://git.psu.edu/genomath/datasets/-
  /raw/main/at_mutants/”, “/idiv/idiv_memory-col0-control-
  gen3_all-contexts_sample-”, 1:5,“.RData”)
temp <− tempfile(fileext = “.RData”)
idiv <− vector(mode = “list”, length = 5)
names(idiv) <− c(“wt3_1”, “wt3_2”, “wt3_3”, “wt3_4”, “wt3_5”)
for (k in 1:5) {
  download.file(url = url[k], destfile = temp)
  load(temp)
  file.remove(temp)
  idiv[[k]] <− jdiv
}
rm(temp, url)
```

The best fitted probability distribution model can be found applying function gofReport:

```
jdiv <− lapply(idiv, function(x) {
  x <− split(x, as.factor(seqnames(x)))
  x<− structure(as.list(x), class = “InfDiv”)
  return(x)
})
Jdiv
d <− c(“Weibull2P”, “Weibull3P”, “Gamma2P”,
  “Gamma3P”, “GGamma3P”, “GGamma4P”)
gof_jd <− lapply(
  jdiv,
  gofReport,
  model = d,
  column = 10L,
  num.cores = 6L,
  alt_models = TRUE,
  r.cv = TRUE,
  output = “all”,
  verbose = FALSE)
```

The same can be done for the memory line dataset.

### 6. Thermodynamic State Variables

The Gibb entropy can be estimated applying function gibb_entropy.

### Wildtype Entropy and Helmholtz Free Energy:

```
url <− paste0(“https://git.psu.edu/genomath/datasets/-
  /raw/main/at_mutants/”, “/gofs/gof-jd-by-chr_memory-col0-
  control-gen3_all-contexts.RData”)
temp <− tempfile(fileext = “.RData”)
download.file(url = url, destfile = temp)
load(temp)
file.remove(temp)
#> [1] TRUE
## Entropy
t(sapply(gof_jd, gibb_entropy))
#>
```

| | | | | | |
|---|---|---|---|---|---|
| #> | 1 | 2 | 3 | 4 | 5 |
| #> wt3_1 | −12.09485 | −13.09161 | −12.85370 | −12.87477 | −12.39782 |
| #> wt3_2 | −12.23864 | −13.20160 | −12.82698 | −12.95472 | −12.44664 |
| #> wt3_3 | −12.58199 | −13.61125 | −13.31159 | −13.40339 | −12.87161 |
| #> wt3_4 | −12.19005 | −13.28911 | −12.88366 | −13.00763 | −12.53417 |
| #> wt3_5 | −13.00950 | −14.07444 | −13.80581 | −13.83110 | −13.33332 |

The Gibb entropy can be estimated applying function helmholtz_free_energy:

t(sapply(gof_jd, helmholtz_free_energy))

| | | | | | |
|---|---|---|---|---|---|
| #> | 1 | 2 | 3 | 4 | 5 |
| #> wt3_1 | 3751.217 | 4060.363 | 3986.574 | 3993.111 | 3845.185 |
| #> wt3_2 | 3795.814 | 4094.477 | 3978.287 | 4017.906 | 3860.326 |
| #> wt3_3 | 3902.303 | 4221.530 | 4128.590 | 4157.063 | 3992.129 |
| #> wt3_4 | 3780.745 | 4121.616 | 3995.866 | 4034.315 | 3887.474 |
| #> wt3_5 | 4034.897 | 4365.187 | 4281.872 | 4289.717 | 4135.330 |

### Memory Line Entropy and Helmholtz Free Energy:

url <− paste0(“https://git.psu.edu/genomath/datasets/-
/raw/main/at_mutants/”,
“/gofs/gof-jd-by-chr_memory-gen3_all-contexts.RData”)
temp <− tempfile(fileext = “.RData”)
download.file(url = url, destfile = temp)
load(temp)
file.remove(temp)
#> [1] TRUE
## Entropy
t(sapply(gof_jd, gibb_entropy))

| | | | | | |
|---|---|---|---|---|---|
| #> | 1 | 2 | 3 | 4 | 5 |
| #> m3_1 | −9.504246 | −10.59310 | −10.36580 | −10.37016 | −9.850248 |
| #> m3_2 | −9.617158 | −10.69061 | −10.53673 | −10.52800 | −10.013634 |
| #> m3_3 | −9.391835 | −10.47471 | −10.26938 | −10.26390 | −9.839075 |
| #> m3_4 | −10.335803 | −11.40691 | −11.29229 | −11.31029 | −10.824549 |
| #> m3_5 | −9.687667 | −10.73626 | −10.53089 | −10.52618 | −10.083295 |

### The Gibb Entropy:

t(sapply(gof_jd, helmholtz_free_energy))

| #> | | 1 | 2 | 3 | 4 | 5 |
|---|---|---|---|---|---|---|
| #> | m3_1 | 2947.742 | 3285.450 | 3214.954 | 3216.304 | 3055.054 |
| #> | m3_2 | 2982.762 | 3315.694 | 3267.967 | 3265.259 | 3105.729 |
| #> | m3_3 | 2912.878 | 3248.730 | 3185.049 | 3183.348 | 3051.589 |
| #> | m3_4 | 3205.649 | 3537.854 | 3502.303 | 3507.888 | 3357.234 |
| #> | m3_5 | 3004.630 | 3329.852 | 3266.155 | 3264.696 | 3127.334 |

### R Script for the Analysis of Entropy Fluctuations

The libraries required and auxiliary functions for the analysis are loaded.

```
library(MethylIT)
library(ggplot2)
library(ggpmisc)
library(dplyr)
## ----- Auxiliary functions ------
lm_egn <− function(x, y) {
  m <− lm(y ~ x + 0)
  eq <− substitute(italic(y) == b %.%
    italic(x)*“,”~~italic(R[abj])\hat{ }2~“=”~r2,
    list(
    b = format(unname(coef(m) [1]), digits = 3),
    r2 = format(summary(m)$adj.r.squared, digits = 3)))
  as.character(as.expression(eq));
}
lm_eqn2 <− function(x, y) {
  m <− lm(y ~ x)
  eq <− substitute(italic(y) == b %.% italic(x) +
    a*“,”~~italic(R[abj])\hat{ }2~“=”~r2,
    list(a = format(unname(coef(m) [1]), digits = 3),
    b = format(unname(coef(m) [2]), digits = 3),
    r2 = format(summary(m)$adj.r.squared, digits = 3)))
  as.character(as.expression(eq));
}
### -------- Memory GOFs -----------
boltz_fact <− function(s) {
  nms <− names(s)
  r <− lapply(seq(s), function(k) {
    r <− data.frame(boltzman_factor(s[[k]], only.sum =
      FALSE))
    r$sample <− rep(nms[k], nrow(r))
    r$chr <− rownames(r)
    rownames(r) <− NULL
    return(r)
  })
  do.call(rbind,r)
}
bfs <− function(s) {
  nms <− names(s)
  r <− lapply(seq(s), function(k) {
    r <− data.frame(boltzman_factor(s[[k]], only.sum =
      FALSE))
    r$chr <− rep(nms[k], nrow(r))
    r$sample <− rownames(r)
    r <− r[−c(9:10),] ## BreastMeta, BreastCancer,
      SquamousCancer,
    rownames(r) <− NULL ## and Adenocarcinoma are removed
    return(r)
  })
  do.call(rbind,r)
}
```

### 1. Arabidopsis Dataset

The Arabidopsis thaliana methylome datasets of msh1 memory and non-memory (normal looking) sibling plants were derived from the msh1 mutant. Basically, as described in reference (1) (main text), a transgene positive plant was self-pollinated and transgene was segregated in subsequent generation. Of transgene null plants, 20% plants displayed delayed in flowering, smaller in size, and lighter green termed as memory phenotype. Memory plants were self-pollinated for six generations and plants from each generation were Bisulfite sequenced.

The dataset generation 2-6 can be accessed with GEOaccession number GSE129303 and GSE118874. The RData files with J-information-divergence and with the best fitted models and corresponding goodness-of-fit used in all the computation reported in the main text are available at PSU GitLab: https://git.psu.edu/genomath/datasets.

```
## ===================== Memory GOFs ======================
boltz_fact <− function(s) {
  nms <− names(s)
  r <− lapply(seq(s), function(k) {
    r <− data.frame(boltzman_factor(s[[k]], only.sum =
      FALSE))
    r$abs_ent <− abs(r$ent)
    r$sample <− rep(nms[k], nrow(r))
    r$chr <− rownames(r)
    rownames(r) <− NULL
    return(r)
  })
  do.call(rbind,r)
}
## ===== Download methylation data from GitLab ========
samples <− c(“memory-col0-control-gen3”,
  “non-memory-gen1”,
  “memory-gen1”,
  “memory-gen2”,
  “memory-gen3”
  “memory-gen4”,
  “memory-gen5”,
  “memory-gen6”,
  “col0-control-met1”,
  “met1”)
url <− paste0(“https://git.psu.edu/genomath/datasets/-
  /raw/main/at_mutants/”, “gofs/gof-jd-by-chr_”,
  samples,“_all-contexts.RData”)
nms <− c(“ctrl”, “nm”, “mm1”, “mm3”, “mm4”, “mm5”, “mm6”,
  “ctrl_met1”, “met1”)
bf <− c( )
for (k in seq(samples)) {
  temp <− tempfile(fileext = “.RData”)
  download.file(url = url[k], destfile = temp)
  load(temp)
  file.remove(temp)
  bf <− rbind(bf, boltz_fact(gof_jd))
}
rm(temp, url)
## To distinguish met1 wildtype control from memmory control
bf$sample[201:220] <− gsub(“wt”, “ctrl_met1”,bf$sample[201:220])
bf$species <− “Arabidopsis”
df <− bf %>% group_by(chr) %>% summarise(means = mean(exp_sum),
              n = n( ), sd =
                sd(exp_sum),
              se = sd/sqrt(n))
df$species <− “Arabidopsis”
df$chr <− paste0(“ch”, 1:5)
dt.0 <− bf %>% group_by(chr,sample) %>% summarise(means =
  mean(exp_sum))
dt.0$species <− “Arabidopsis”
```

### 2. Plotting Linear Regression

bf. <− bf[ !grepl(“met1”, bf$sample), ]
bf.0 <− bf[ grepl(“met1”, bf$sample), ]
bf.1 <− bf[ grepl(“ctrl_met1”, bf$sample), ]
lm_bf <− lm(abs_ent ~ nu, data = bf.)
summary(lm_bf)
#>

| | |
|---|---|
| #> | Call: |
| #> | lm(formula = abs_ent ~ nu, data = bf.) |

#>

| #> | Residuals: | | | | |
|---|---|---|---|---|---|
| #> | Min | 1Q | Median | 3Q | Max |
| #> | −0.056783 | −0.020092 | −0.004458 | 0.018324 | 0.085323 |

#>

| #> | Coefficients: | | | | |
|---|---|---|---|---|---|
| #> | | Estimate | Std. Error | t value | Pr(>\|t\|) |
| #> | (Intercept) | 2.34582 | 0.01019 | 230.2 | <2e−16 *** |
| #> | nu | −7.65431 | 0.08357 | −91.59 | <2e−16 *** |

| | |
|---|---|
| #> | --- |
| #> | Signif. codes: 0 ‘***’ 0.001 ‘**’ 0.01 ‘*’ 0.05 ‘.’ 0.1 ‘ ’ |
| | 1 |

#>

| | |
|---|---|
| #> | Residual standard error: 0.02813 on 198 degrees of freedom |
| #> | Multiple R-squared: 0.9769, Adjusted R-squared: 0.9768 |
| #> | F-statistic: 8389 on 1 and 198 DF, p-value: < 2.2e−16 |

p <− ggplot(bf., aes(x = nu, y = abs_ent)) +

| | |
|---|---|
| | geom_point(data = bf.) + |
| | geom_smooth(data = bf., formula = y ~ x, method=lm , se = |
| | TRUE) + |
| | theme_light(base_family = “serif”) + |
| | theme(text = element_text(size = 20, family = “serif”)) + |
| | geom_text(x = 0.11, y = 1.18, label = lm_eqn2(x = bf.$nu, |
| | y = bf.$abs_ent), |
| | parse = TRUE, family = “serif”, |
| | size = 4) |
| p0 | <− ggplot(bf.0, aes(x = nu, y = abs_ent)) + |
| | geom_point(color=“red”) + |
| | geom_smooth(data = bf.1, formula = y ~ x, method=lm , se |
| | = TRUE, fullrange = F) + |
| | geom_point(data = bf.1) + |
| | theme_light(base_family = “serif”) + |
| | theme(axis.title.x = element_blank( ), |
| | axis.title.y = element_blank( ), |
| | text = element_text(size = 10, family = “serif”)) + |
| | geom_text(x = 0.45, y = 0.35, label = lm_eqn2(x=bf.1$nu, |
| | y=bf.1$abs_ent), |

```
  parse = TRUE, family = “serif”,
  size = 3)
```

p + annotation_custom(grob = ggplotGrob(p0),

```
      xmin = 0.125, xmax = 0.175,
      ymin = 1.41, ymax = 1.82 )
```

p <− ggplot(bf., aes(x = nu, y = exp_ent)) +

```
  geom_point(data = bf.) +
  geom_smooth(data = bf., formula = y ~ x + 0, method=lm ,
      se = TRUE) +
  theme_light(base_family = “serif”) +
  theme(text = element_text(size = 20, family = “serif”)) +
  geom_text(x = 0.14, y = 0.22, label = lm_eqn(x=bf.$nu,
```

y=bf.$exp_ent),

| | |
|---|---|
| | parse = TRUE, family = “serif”, |
| | size = 4) |
| p0 | <− ggplot(bf.0, aes(x = nu, y = exp_ent)) + |
| | geom_point(color=“red”) + |
| | geom_smooth(data = bf.1, formula = y ~ x, |
| | method=lm , se = TRUE, fullrange = F) + |
| | geom_point(data = bf.1) + |
| | theme_light(base_family = “serif”) + |
| | theme(axis.title.x = element_blank( ), |
| | axis.title.y = element_blank( ), |
| | text = element_text(size = 10, family = “serif”)) + |
| | geom_text(x = 0.48, y = 0.52, label = lm_eqn(x=bf.1$nu, |
| | y=bf.1$exp_ent), |

```
  parse = TRUE, family = “serif”,
  size = 3, color = “blue”)
```

p + annotation_custom(grob = ggplotGrob(p0),

```
  xmin = 0.078, xmax = 0.125,
  ymin = 0.26, ymax = 0.36 )
```

### Regression Analysis

lm_bf <− lm(exp_ent ~ exp_nu, data = bf.)
summary(lm_bf)
#>

| | |
|---|---|
| #> | Call: |
| #> | lm(formula = exp_ent ~ exp_nu, data = bf.) |

#>

| #> | Residuals: | | | | |
|---|---|---|---|---|---|
| #> | Min | 1Q | Median | 3Q | Max |
| #> | −0.014470 | −0.003848 | 0.000030 | 0.003861 | 0.016206 |

#>

| #> | Coefficients: | | | | |
|---|---|---|---|---|---|
| #> | | Estimate | Std. Error | t value | Pr(>\|t\|) |
| #> | (Intercept) | 2.14218 | 0.01586 | 135.0 | <2e−16 *** |
| #> | exp_nu | −2.13947 | 0.01787 | −119.7 | <2e−16 *** |

| | |
|---|---|
| #> | --- |
| #> | Signif. codes: 0 ‘***’ 0.001 ‘**’ 0.01 ‘*’ 0.05 ‘.’ 0.1 ‘ ’ |
| | 1 |

#>

| | |
|---|---|
| #> | Residual standard error: 0.005313 on 198 degrees of freedom |
| #> | Multiple R-squared: 0.9864, Adjusted R-squared: 0.9863 |
| #> | F-statistic: 1.434e+04 on 1 and 198 DF, p-value: < 2.2e−16 |

p <− ggplot(bf.0, aes(x = exp_nu, y = exp_ent)) +

| | |
|---|---|
| | geom_point(data = bf.) + |
| | geom_smooth(data = bf., formula = y ~ x, method=lm , se = |
| | TRUE) + |
| | theme_light(base_family = “serif”) + |
| | theme(text = element_text(size = 20, family = “serif”)) + |
| | geom_text(x = 0.87, y = 0.18, |
| | label = lm_eqn(x=bf.0$exp_nu, y=bf.0$exp_ent), |
| | parse = TRUE, family = “serif”, |
| | size = 4) |
| p0 | <− ggplot(bf.0, aes(x = exp_nu, y = exp_ent)) + |
| | geom_point(color=“red”) + |
| | geom_smooth(data = bf.1, formula = y ~ x, |
| | method=lm , se = TRUE, fullrange = F) + |
| | geom_point(data = bf.1) + |
| | theme_light(base_family = “serif”) + |
| | theme(axis.title.x = element_blank( ), |
| | axis.title.y = element_blank( ), |
| | text = element_text(size = 10, family = “serif”)) + |
| | geom_text(x = 0.65, y = 0.69, label = |
| | lm_eqn(x=bf.1$exp_nu, y=bf.1$exp_ent), |

```
  parse = TRUE, family = “serif”,
  size = 3)
```

p + annotation_custom(grob = ggplotGrob(p0),

```
  xmin = 0.88, xmax = 0.924,
  ymin = 0.26, ymax = 0.36 )
```

### 3. Cancer Dataset

## ===== Download methylation data from GitLab ========
url <− paste0(“https://git.psu.edu/genomath/datasets/-/raw/”,

```
  “main/cancer_data/gofs/jdiv_gof_per-chr(10)_gwf_cancer_05-
  16-22.RData”)
```

temp <− tempfile(fileext = “.RData”)
download.file(url = url, destfile = temp)
load(temp)
file.remove(temp); rm(temp, url)

| | |
|---|---|
| #> | [1] TRUE |

bfs <− function(s) {

```
  nms <− names(s)
  r <− lapply(seq(s), function(k) {
      r <− data.frame(boltzman_factor(s[[k]], only.sum =
```

FALSE))

```
      r$abs_ent <− abs(r$ent)
      r$chr <− rep(nms[k], nrow(r))
      r$sample <− rownames(r)
      r <− r[−c(9:10),] ## BreastMeta, BreastCancer,
```

SquamousCancer,

```
      rownames(r) <− NULL ## and Adenocarcinoma are removed
      return(r)
  })
  do.call(rbind,r)
```

}
bf2 <− bfs(gofs)
bf2$species <− “human”
dt <− bf2 %>% group_by(chr) %>% summarise(means =

mean(exp_sum),

```
  n = n( ), sd = sd(exp_sum),
  se = sd / sqrt(n))
```

dt$chr <− factor(dt$chr,

levels = c( “3”,“4”,“5”,“6”,“7”, “8”,“9”,“10”,“17”,“22”))

dt$species <− “human”
dt. <− bf2 %>% group_by(chr,sample) %>% summarise(means =

mean(exp_sum))

dt.$species <− “human”
bf3 <− bf2[ bf2$sample != “BreastMeta” & bf2$sample !=

“BreastCancer”, ]

bf3 <− bf3[ ! grepl(“hesc”, bf3$sample), ] ## Without stem cells
bf3. <− bf2[ grepl(“hesc”, bf2$sample), ] ## Only stem cells
lm_bf2 <− lm(abs_ent ~ nu, data = bf3)
summary(lm_bf2)
#>

| | |
|---|---|
| #> | Call: |
| #> | lm(formula = abs_ent ~ nu, data = bf3) |

#>

| #> | Residuals: | | | | |
|---|---|---|---|---|---|
| #> | Min | 1Q | Median | 3Q | Max |
| #> | −0.38714 | −0.17694 | 0.01535 | 0.13973 | 0.58889 |

#>

| | |
|---|---|
| #> | Coefficients: |

#>

| #> | | Estimate | Std. Error | t value | Pr(>\|t\|) |
|---|---|---|---|---|---|
| #> | (Intercept) | 2.02504 | 0.03244 | 62.42 | <2e−16 *** |
| #> | nu | −3.19009 | 0.11274 | −28.30 | <2e−16 *** |

| | |
|---|---|
| #> | --- |
| #> | Signif. codes: 0 ‘***’ 0.001 ‘**’ 0.01 ‘*’ 0.05 ‘.’ 0.1 ‘ ’ |
| | 1 |

#>

| | |
|---|---|
| #> | Residual standard error: 0.1845 on 78 degrees of freedom |
| #> | Multiple R-squared: 0.9112, Adjusted R-squared: 0.9101 |
| #> | F-statistic: 800.7 on 1 and 78 DF, p-value: < 2.2e−16 |

ggplot(bf2, aes(x = nu, y = abs_ent)) +

```
  geom_point(color=“red”) +
  geom_smooth(method=lm , color=“red”, se = TRUE) +
  geom_point(data = bf3) +
  geom_smooth(data = bf3, method=lm , se = TRUE) +
  geom_point(data = bf3., color = “magenta”) +
  theme_light(base_family = “serif”) +
  theme(text = element_text(size = 10, family = “serif”)) +
  geom_text(x = 0.4, y = −0.5, label = lm_eqn2(x=bf3$nu,
```

y=bf3$abs_ent),

```
      parse = TRUE, family = “serif”,
      color = “blue”, size = 4) +
  geom_text(x = 0.75, y = 1.5, label = lm_eqn2(x=bf2$nu,
```

y=bf2$abs_ent),

```
      parse = TRUE, family = “serif”,
      color = “red”, size = 4)
```

ggplot(bf2, aes(x = nu, y = exp_ent)) +

```
  geom_point(color=“red”) +
  geom_smooth(method=lm , formula = y ~ x + 0, color=“red”,
      se = TRUE) +
  geom_point(data = bf3) +
  geom_smooth(data = bf3, method=lm , se = TRUE) +
  geom_point(data = bf3., color = “magenta”) +
  theme_light(base_family = “serif”) +
  theme(text = element_text(size = 10, family = “serif”)) +
  geom_text(x = 0.7, y = 0.2, label = lm_eqn2(x=bf2$nu,
```

y=bf2$exp_ent),

```
      parse = TRUE, family = “serif”,
      size = 4, color = “red”) +
  geom_text(x = 0.5, y = 1.1, label = lm_eqn2(x=bf3$nu,
```

y=bf3$exp_ent),

```
      parse = TRUE, family = “serif”,
      size = 4, color = “blue”)
```

### 4. Plotting Linear Regression

```
ggplot(bf2, aes(x = exp_nu, y = exp_ent)) +
  geom_point(color=“red”) +
  geom_smooth(method=lm , color=“red”, se = TRUE) +
  geom_point(data = bf3) +
  geom_smooth(data = bf3, method=lm , se = TRUE) +
  geom_point(data = bf3., color = “magenta”) +
  theme_light(base_family = “serif”) +
  theme(text = element_text(size = 20, family = “serif”)) +
  geom_text(x = 0.45, y = 0.2, label =
      lm_eqn2(x=bf2$exp_nu, y=bf2$exp_ent),
    parse = TRUE, family = “serif”,
    color = “red”, size = 4) +
  geom_text(x = 0.75, y = 0.95, label =
      lm_eqn2(x=bf3$exp_nu, y=bf3$exp_ent),
    parse = TRUE, family = “serif”,
    color = “blue”, size = 4)
```

### 5. Barplot

### Reorganizing Datasets:

library(ggplot2)
library(ggpmisc)
dat <− rbind(df, dt)

| | |
|---|---|
| st | <− dat %>% group_by(species) %>% |

```
summarize(mean = round(mean(means), 3),
      n = n( ), sd = round(sd(means), 3),
      se = round(sd / sqrt(n), 3))
```

colnames(st) <− c(“species”, “ mean ”, “ sample.size ”,“

standard.dev ”, “ standard.Err ” )

chr <− c(“ch1”, “ch2”, “ch3”, “ch4”, “ch5”,

“3”,“4”,“5”,“6”,“7”,
“8”,“9”,“10”,“17”,“22”)

dat

| #> | # | A tibble: 15 × 6 | | | | | |
|---|---|---|---|---|---|---|---|
| #> | | chr | means | n | sd | se | species |
| #> | | <chr> | <dbl> | <int> | <dbl> | <dbl> | <chr> |
| #> | 1 | ch1 | 1.17 | 47 | 0.0764 | 0.0111 | Arabidopsis |
| #> | 2 | ch2 | 1.15 | 47 | 0.0819 | 0.0119 | Arabidopsis |
| #> | 3 | ch3 | 1.16 | 47 | 0.0817 | 0.0119 | Arabidopsis |
| #> | 4 | ch4 | 1.16 | 47 | 0.0844 | 0.0123 | Arabidopsis |
| #> | 5 | ch5 | 1.17 | 47 | 0.0805 | 0.0117 | Arabidopsis |
| #> | 6 | 10 | 1.17 | 13 | 0.187 | 0.0519 | human |
| #> | 7 | 17 | 1.15 | 13 | 0.151 | 0.0418 | human |
| #> | 8 | 22 | 1.19 | 13 | 0.128 | 0.0355 | human |
| #> | 9 | 3 | 1.18 | 13 | 0.159 | 0.0440 | human |
| #> | 10 | 4 | 1.17 | 13 | 0.194 | 0.0539 | human |
| #> | 11 | 5 | 1.17 | 13 | 0.191 | 0.0529 | human |
| #> | 12 | 6 | 1.19 | 13 | 0.138 | 0.0381 | human |
| #> | 13 | 7 | 1.15 | 13 | 0.152 | 0.0421 | human |
| #> | 14 | 8 | 1.19 | 13 | 0.198 | 0.0548 | human |
| #> | 15 | 9 | 1.19 | 13 | 0.137 | 0.0380 | human |

### The Barplot:

```
p <− ggplot(dat, aes(x = chr, y = means, fill = species)) +
  scale_x_discrete(limits = chr) +
  geom_bar(stat=“identity”, position=position_dodge( )) +
  geom_errorbar(aes(ymin = means − sd, ymax = means + sd),
    width =.2, position=position_dodge(.9)) +
  geom_hline(yintercept = 1., linetype=“dashed”, color =
    “red”) +
  xlab(“Chromosome”) + ylab(“Sum of Boltzmann's factors”) +
  # geom_text(aes(1.5, 1., label = 1., vjust = −1), family =
    “serif”) +
  geom_text(aes(label = n), colour = “white”, fontface =
    “bold”, nudge_y = −0.5, family = “serif”) +
  theme_light(base_family = “serif”, base_size = 14) +
  theme(text = element_text(size = 16, family = “serif”),
    legend.margin=margin(4,4,4,4),
    legend.box.spacing = margin(0.5),
    legend.position=“bottom”) +
  annotate(geom = “table”,
    x = 15,
    y = 1.8,
    label = list(st),
    size = 4,
    family = “serif”)
p + scale_fill_brewer(palette=“Paired”)
```

### Disruption of Breast Cancer and Stem Cells:

```
st1. <− data.frame(dt.) [ is.element(dt.$sample, c(“Brain”,
  “Breast”, “Colon”, “Lung”)),
]
st1. <− st1. %>% group_by(sample) %>%
  summarize(mean = round(mean(means),3),
    n = n ( ), sd = round(sd(means),3),
    se = round(sd / sqrt(n), 3))
ggplot(dt., aes(x = sample, y = means, fill = sample)) +
  geom_boxplot(color = “red”,shape=21) +
  theme_light(base_family = “serif”, base_size = 14) +
  theme(text = element_text(size = 16, family = “serif”),
    axis.text.x = element_text(angle = 45, vjust = 1,
      hjust=1),
    legend.margin−margin(4,4,4,4),
    legend.box.spacing = margin(0.5)) +
    annotate(geom = “table”,
      x = 5.2,
      y = 0.6,
      label = list(st1.),
      size = 4,
      family = “serif”)
```

### R Script for Generating Group Differences in Arabidopsis Memory Line Based Entropy

The group comparisons ‘control’ versus mash1-memory-lines are presented here. We are interested in to learn whether the Gibb entropy (gent) of methylation variation, measured with respect to some reference state, coincides with observable phenotypic change. gent was estimated in Arabidopsis thaliana Col-0 ecotypes (wildtype controls, WT), the methyltransferase mutant met1 (1), and first and third-generation heritable epigenetic memory states (nm1, mm1, and mm3), which derive as epigenetically modified progeny from a parental line following suppression of MSH1 expression.

```
library(MethylIT)
#> Warning: replacing previous import
  ‘lifecycle::last_{—}warnings' by
#> ‘rlang::last_{—}warnings' when loading ‘tibble’
#> Warning: replacing previous import
  ‘lifecycle::last_{—}warnings' by
#> ‘rlang::last_{—}warnings' when loading ‘pillar’
library(lmerTest)
library(lme4)
library(reshape2)
```

Next, an auxiliary function to format the datasets into ‘data.frames’

```
gent <− function(x) {
  x <− t( sapply(x, gibb_entropy))
  colnames(x) <− paste0(“chr”, colnames(x))
  x = melt(x)
  colnames(x) <− c(“sample”, “chr”, “entropy”)
  x$chr <− factor(x$chr)
  return(x)
}
```

The RData files with nonlinear fit models can be downloaded from GitLab using the following script:

## ===== Download methylation data from GitLab ========
samples <− c(“memory-col0-control-gen3”,

“non-memory-gen1”,
“memory-gen1”,
“memory-gen3”,
“col0-control-met1”,
“met1”)

url <− paste0(“https://git.psu.edu/genomath/datasets/-

/raw/main/at_mutants/”, “gofs/gof-jd-by-chr_”,
samples,“_all-contexts.RData”)

nms <− c(“ctrl”, “nm”, “mm1”, “mm3_”, “ctrl_met1”, “met1”)
gofs <− vector(mode = “list”, length = 6)
names(gofs) <− nms
for (k in seq(samples)) {

```
  temp <− tempfile(fileext = “.RData”)
  download.file(url = url[k], destfile = temp)
  load( temp)
  file.remove(temp)
  gent_wt_<− gent(gof_jd)
  gent_wt$group <− nms[ k ]
  gent_wt$group <− factor(gent_wt$group)
  gofs[[k]] <− gent_wt
```

}
rm(temp, url)
gofs

| #> | $ctrl | | | | |
|---|---|---|---|---|---|
| #> | | sample | chr | entropy | group |
| #> | 1 | wt3_{—}1 | chr1 | −12.09485 | ctrl |
| #> | 2 | wt3_{—}2 | chr1 | −12.23864 | ctrl |
| #> | 3 | wt3_{—}3 | chr1 | −12.58199 | ctrl |
| #> | 4 | wt3_{—}4 | chr1 | −12.19005 | ctrl |
| #> | 5 | wt3_{—}5 | chr1 | −13.00950 | ctrl |
| #> | 6 | wt3_{—}1 | chr2 | −13.09161 | ctrl |
| #> | 7 | wt3_{—}2 | chr2 | −13.20160 | ctrl |
| #> | 8 | wt3_{—}3 | chr2 | −13.61125 | ctrl |
| #> | 9 | wt3_{—}4 | chr2 | −13.28911 | ctrl |
| #> | 10 | wt3_{—}5 | chr2 | −14.07444 | ctrl |
| #> | 11 | wt3_{—}1 | chr3 | −12.85370 | ctrl |
| #> | 12 | wt3_{—}2 | chr3 | −12.82698 | ctrl |
| #> | 13 | wt3_{—}3 | chr3 | −13.31159 | ctrl |
| #> | 14 | wt3_{—}4 | chr3 | −12.88366 | ctrl |
| #> | 15 | wt3_{—}5 | chr3 | −13.80581 | ctrl |
| #> | 16 | wt3_{—}1 | chr4 | −12.87477 | ctrl |
| #> | 17 | wt3_{—}2 | chr4 | −12.95472 | ctrl |
| #> | 18 | wt3_{—}3 | chr4 | −13.40339 | ctrl |
| #> | 19 | wt3_{—}4 | chr4 | −13.00763 | ctrl |
| #> | 20 | wt3_{—}5 | chr4 | −13.83110 | ctrl |
| #> | 21 | wt3_{—}1 | chr5 | −12.39782 | ctrl |
| #> | 22 | wt3_{—}2 | chr5 | −12.44664 | ctrl |
| #> | 23 | wt3_{—}3 | chr5 | −12.87161 | ctrl |
| #> | 24 | wt3_{—}4 | chr5 | −12.53417 | ctrl |
| #> | 25 | wt3_{—}5 | chr5 | −13.33332 | ctrl |

#>

| #> | $nm | | | | |
|---|---|---|---|---|---|
| #> | | sample | chr | entropy | group |
| #> | 1 | nm1_{—}1 | chr1 | −10.51669 | nm |
| #> | 2 | nm1_{—}2 | chr1 | −10.34410 | nm |
| #> | 3 | nm1_{—}3 | chr1 | −13.42377 | nm |
| #> | 4 | nm1_{—}4 | chr1 | −10.33168 | nm |
| #> | 5 | nm1_{—}5 | chr1 | −14.45793 | nm |
| #> | 6 | nm1_{—}1 | chr2 | −11.67142 | nm |
| #> | 7 | nm1_{—}2 | chr2 | −11.46055 | nm |
| #> | 8 | nm1_{—}3 | chr2 | −14.23364 | nm |
| #> | 9 | nm1_{—}4 | chr2 | −11.42840 | nm |
| #> | 10 | nm1_{—}5 | chr2 | −14.97243 | nm |
| #> | 11 | nm1_{—}1 | chr3 | −11.42988 | nm |
| #> | 12 | nm1_{—}2 | chr3 | −11.19313 | nm |
| #> | 13 | nm1_{—}3 | chr3 | −14.12629 | nm |
| #> | 14 | nm1_{—}4 | chr3 | −11.16044 | nm |
| #> | 15 | nm1_{—}5 | chr3 | −15.00178 | nm |
| #> | 16 | nm1_{—}1 | chr4 | −11.44687 | nm |
| #> | 17 | nm1_{—}2 | chr4 | −11.20523 | nm |
| #> | 18 | nm1_{—}3 | chr4 | −14.17518 | nm |
| #> | 19 | nm1_{—}4 | chr4 | −11.19233 | nm |
| #> | 20 | nm1_{—}5 | chr4 | −14.80361 | nm |
| #> | 21 | nm1_{—}1 | chr5 | −10.97023 | nm |
| #> | 22 | nm1_{—}2 | chr5 | −10.75758 | nm |
| #> | 23 | nm1_{—}3 | chr5 | −13.76125 | nm |
| #> | 24 | nm1_{—}4 | chr5 | −10.73998 | nm |
| #> | 25 | nm1_{—}5 | chr5 | −14.61423 | nm |
| #> | $mm1 | | | | |
| #> | | sample | chr | entropy | group |
| #> | 1 | m1_{—}1 | chr1 | −12.452386 | mm1 |
| #> | 2 | m1_{—}2 | chr1 | −13.169982 | mm1 |
| #> | 3 | m1_{—}3 | chr1 | −10.484701 | mm1 |
| #> | 4 | m1_{—}4 | chr1 | −10.087015 | mm1 |
| #> | 5 | m1_{—}5 | chr1 | −9.969216 | mm1 |
| #> | 6 | m1_{—}1 | chr2 | −13.384541 | mm1 |
| #> | 7 | m1_{—}2 | chr2 | −14.111095 | mm1 |
| #> | 8 | m1_{—}3 | chr2 | −11.578029 | mm1 |
| #> | 9 | m1_{—}4 | chr2 | −11.177138 | mm1 |
| #> | 10 | m1_{—}5 | chr2 | −11.103592 | mm1 |
| #> | 11 | m1_{—}1 | chr3 | −13.152839 | mm1 |
| #> | 12 | m1_{—}2 | chr3 | −13.933727 | mm1 |
| #> | 13 | m1_{—}3 | chr3 | −11.390940 | mm1 |
| #> | 14 | m1_{—}4 | chr3 | −10.971958 | mm1 |
| #> | 15 | m1_{—}5 | chr3 | −10.818339 | mm1 |
| #> | 16 | m1_{—}1 | chr4 | −13.134270 | mm1 |
| #> | 17 | m1_{—}2 | chr4 | −13.978335 | mm1 |
| #> | 18 | m1_{—}3 | chr4 | −11.368858 | mm1 |
| #> | 19 | m1_{—}4 | chr4 | −10.981584 | mm1 |
| #> | 20 | m1_{—}5 | chr4 | −10.851584 | mm1 |
| #> | 21 | m1_{—}1 | chr5 | −12.807018 | mm1 |
| #> | 22 | m1_{—}2 | chr5 | −13.578820 | mm1 |
| #> | 23 | m1_{—}3 | chr5 | −10.946872 | mm1 |
| #> | 24 | m1_{—}4 | chr5 | −10.484795 | mm1 |
| #> | 25 | m1_{—}5 | chr5 | −10.297509 | mm1 |

#>

| #> | $mm3_{—} | | | | |
|---|---|---|---|---|---|
| #> | | sample | chr | entropy | group |
| #> | 1 | m3_{—}1 | chr1 | −9.504246 | mm3 |
| #> | 2 | m3_{—}2 | chr1 | −9.617158 | mm3 |
| #> | 3 | m3_{—}3 | chr1 | −9.391835 | mm3 |
| #> | 4 | m3_{—}4 | chr1 | −10.335803 | mm3 |
| #> | 5 | m3_{—}5 | chr1 | −9.687667 | mm3 |
| #> | 6 | m3_{—}1 | chr2 | −10.593100 | mm3 |
| #> | 7 | m3_{—}2 | chr2 | −10.690615 | mm3 |
| #> | 8 | m3_{—}3 | chr2 | −10.474706 | mm3 |
| #> | 9 | m3_{—}4 | chr2 | −11.406912 | mm3 |
| #> | 10 | m3_{—}5 | chr2 | −10.736263 | mm3 |
| #> | 11 | m3_{—}1 | chr3 | −10.365805 | mm3 |
| #> | 12 | m3_{—}2 | chr3 | −10.536732 | mm3 |
| #> | 13 | m3_{—}3 | chr3 | −10.269382 | mm_{—} |
| #> | 14 | m3_{—}4 | chr3 | −11.292288 | mm3 |
| #> | 15 | m3_{—}5 | chr3 | −10.530887 | mm3 |
| #> | 16 | m3_{—}1 | chr4 | −10.370156 | mm3 |
| #> | 17 | m3_{—}2 | chr4 | −10.527998 | mm3 |
| #> | 18 | m3_{—}3 | chr4 | −10.263898 | mm3 |
| #> | 19 | m3_{—}4 | chr4 | −11.310295 | mm3 |
| #> | 20 | m3_{—}5 | chr4 | −10.526185 | mm3 |
| #> | 21 | m3_{—}1 | chr5 | −9.850248 | mm3 |
| #> | 22 | m3_{—}2 | chr5 | −10.013634 | mm3 |
| #> | 23 | m3_{—}3 | chr5 | −9.839075 | mm3 |
| #> | 24 | m3_{—}4 | chr5 | −10.824549 | mm3 |
| #> | 25 | m3_{—}5 | chr5 | −10.083295 | mm3 |

#>

| #> | $ctrl_{—}met1 | | | | |
|---|---|---|---|---|---|
| #> | | sample | chr | entropy | group |
| #> | 1 | wt_{—}1 | chr1 | −3.751485 | ctrl_{—}met1 |
| #> | 2 | wt_{—}2 | chr1 | −5.876468 | ctrl_{—}met1 |
| #> | 3 | wt_{—}3 | chr1 | −5.869031 | ctrl_{—}met1 |
| #> | 4 | wt_{—}4 | chr1 | −5.993948 | ctrl_{—}met1 |
| #> | 5 | wt_{—}1 | chr2 | −4.060561 | ctrl_{—}met1 |
| #> | 6 | wt_{—}2 | chr2 | −6.242450 | ctrl_{—}met1 |
| #> | 7 | wt_{—}3 | chr2 | −6.215879 | ctrl_{—}met1 |
| #> | 8 | wt_{—}4 | chr2 | −6.346747 | ctrl_{—}met1 |
| #> | 9 | wt_{—}1 | chr3 | −3.957873 | ctrl_{—}met1 |
| #> | 10 | wt_{—}2 | chr3 | −6.163961 | ctrl_{—}met1 |
| #> | 11 | wt_{—}3 | chr3 | −6.070257 | ctrl_{—}met1 |
| #> | 12 | wt_{—}4 | chr3 | −6.177509 | ctrl_{—}met1 |
| #> | 13 | wt_{—}1 | chr4 | −3.738039 | ctrl_{—}met1 |
| #> | 14 | wt_{—}2 | chr4 | −5.959253 | ctrl_{—}met1 |
| #> | 15 | wt_{—}3 | chr4 | −5.895751 | ctrl_{—}met1 |
| #> | 16 | wt_{—}4 | chr4 | −5.994613 | ctrl_{—}met1 |
| #> | 17 | wt_{—}1 | chr5 | −3.700360 | ctrl_{—}met1 |
| #> | 18 | wt_{—}2 | chr5 | −5.810828 | ctrl_{—}met1 |
| #> | 19 | wt_{—}3 | chr5 | −5.727038 | ctrl_{—}met1 |
| #> | 20 | wt_{—}4 | chr5 | −5.889276 | ctrl_{—}met1 |

#>

| #> | $met1 | | | | |
|---|---|---|---|---|---|
| #> | | sample | chr | entropy | group |
| #> | 1 | met1_{—}1 | chr1 | 2.183079 | met1 |
| #> | 2 | met1_{—}2 | chr1 | 1.199392 | met1 |
| #> | 3 | met1_{—}3 | chr1 | 2.032322 | met1 |
| #> | 4 | met1_{—}1 | chr2 | 2.128942 | met1 |
| #> | 5 | met1_{—}2 | chr2 | 1.126149 | met1 |
| #> | 6 | met1_{—}3 | chr2 | 1.993428 | met1 |
| #> | 7 | met1_{—}1 | chr3 | 2.065479 | met1 |
| #> | 8 | met1_{—}2 | chr3 | 1.072355 | met1 |
| #> | 9 | met1_{—}3 | chr3 | 1.923155 | met1 |
| #> | 10 | met1_{—}1 | chr4 | 1.980010 | met1 |
| #> | 11 | met1_{—}2 | chr4 | 1.004305 | met1 |
| #> | 12 | met1_{—}3 | chr4 | 1.847710 | met1 |
| #> | 13 | met1_{—}1 | chr5 | 2.085467 | met1 |
| #> | 14 | met1_{—}2 | chr5 | 1.107616 | met1 |
| #> | 15 | met1_{—}3 | chr5 | 1.945829 | met1 |

### 1. Linear and Generalized Linear Models for Memory Line 1rt Generation

gent_wt_mm1 <− rbind(gofs$ctrl, gofs$mm1)

lm_wt_mm1 <− lmer(formula = entropy ~ group + (1|chr),

data = gent_wt_mm1)

summary(lm_wt_mm1)

| | |
|---|---|
| #> | Linear mixed model fit by REML. t-tests use Satterthwaite's |
| | method [ |
| #> | lmerModLmerTest] |
| #> | Formula: entropy ~ group +(1 \| chr) |
| #> | Data: gent_{—}wt_{—}mm1 |

#>

| | |
|---|---|
| #> | REML criterion at convergence: 144.9 |

#>

| | |
|---|---|
| #> | Scaled residuals: |
| #> | Min 1Q Median 3Q Max |
| #> | −2.0750 −0.6688 0.1694 0.6279 1.6312 |

#>

| #> | Random effects: | | | |
|---|---|---|---|---|
| #> | Groups | Name | Variance | Std. Dev. |
| #> | chr | (Intercept) | 0.07153 | 0.2675 |

| | | | |
|---|---|---|---|
| #> | Residual | 1.00269 | 1.0013 |

| | |
|---|---|
| #> | Number of obs: 50, groups: chr, 5 |

#>

| #> | Fixed effects: | | | | | |
|---|---|---|---|---|---|---|
| #> | | Estimate | Std. Error | df | t value | Pr(>\|t\|) |
| # | (Intercept) | −12.9888 | 0.2333 | 9.7303 | −55.682 | 1.63e−13 *** |
| #> | groupmm1 | 1.1402 | 0.2832 | 44.0000 | 4.026 | 0.000221 *** |

| | |
|---|---|
| #> | --- |
| #> | Signif. codes: 0 ‘***’ 0.001 ‘**’ 0.01 ‘*’ 0.05 ‘.’ 0.1 ‘ ’ |
| | 1 |

#>

| | |
|---|---|
| #> | Correlation of Fixed Effects: |

| | |
|---|---|
| #> | (Intr) |

| | |
|---|---|
| #> | groupmm1 −0.607 |

anova(lm wt_{—}mm1)

| #> | Type III Analysis of Variance Table with Satterthwaite's | | | | | | |
|---|---|---|---|---|---|---|---|
| | method | | | | | | |
| #> | | Sum Sq | Mean Sq | NumDF | DenDF | F value | Pr(>F) |
| #> | group | 16.25 | 16.25 | 1 | 44 | 16.207 | 0.0002207 * |

| | |
|---|---|
| #> | −−− |
| #> | Signif. codes: 0 ‘***’ 0.001 ‘**’ 0.01 ‘*’ 0. 05 ‘.’ 0.1 ‘ ’ |
| | 1 |

### 2. Linear and Generalized Linear Models for Non-Memory Line

gent_wt_nm <− rbind (gofs$ctrl, gofs$nm)
lm_wt_nm <− lmer(formula = entropy ~ group + (1|chr),

data = gent_wt_nm)

| | |
|---|---|
| #> | boundary(singular)fit: see ?isSingular |

summary(lm_{—}wt_{—}nm)

| | |
|---|---|
| #> | Linear mixed model fit by REML. t-tests use Satterthwaite's |
| | method [ |
| #> | lmerModLmerTest] |
| #> | Formula: entropy ~ group +(1 \| chr) |
| #> | Data: gent_{—}wt_{—}nm |

#>

| | |
|---|---|
| #> | REML criterion at convergence: 165.3 |

#>

| #> | Scaled residuals: | | | | | |
|---|---|---|---|---|---|---|
| #> | Min 1Q Median 3Q Max | | | | | |
| #> | −2.07391 −0.60705 0.09134 0.73194 1.61570 | | | | | |
| #> | | | | | | |
| #> | Random effects: | | | | | |
| #> | Groups Name | | Variance | Std. Dev | | |
| #> | chr (Intercept) | | 5.322e−15 | 7.295e−08 | | |
| #> | Residual | | 1.602e+00 | 1.266e+00 | | |
| #> | Number of obs: 50, groups: chr, 5 | | | | | |
| #> | | | | | | |
| #> | Fixed effects: | | | | | |
| #> | | Estimate | Std. Error | df | t value | Pr(>\|t\|) |
| #> | (Intercept) | −12.9888 | 0.2531 | 48.000 | −51.31 | <2e−16 *** |
| #> | groupnm | 0.6121 | 0.3580 | 48.000 | 1.71 | 0.0938 . |
| #> | --- | | | | | |
| #> | Signif. codes: 0 ‘***’ 0.001 ‘**’ 0.01 ‘*’ 0.05 ‘.’ 0.1 ‘ ’ | | | | | |
| | 1 | | | | | |
| #> | | | | | | |

| #> | Correlation of Fixed Effects: | |
|---|---|---|
| #> | | (Intr) |

| | |
|---|---|
| #> | groupnm −0.707 |
| #> | optimizer(nloptwrap)convergence code: 0(OK) |
| #> | boundary(singular)fit: see ?isSingular |

anova(lm_{—}wt_{—}nm)

| #> | Type III Analysis of Variance Table with Satterthwaite's | | | | | | | |
|---|---|---|---|---|---|---|---|---|
| | method | | | | | | | |
| #> | | Sum Sq | Mean Sq | NumDF | DenDF | F value | Pr(>F) | |
| #> | group | 4.6826 | 4.6826 | 1 | 48 | 2.9228 | 0.0938 | . |
| #>--- | | | | | | | | |

| | |
|---|---|
| #> | Signif. codes: 0 ‘***’ 0.001 ‘**’ 0.01 ‘*’ 0.05 ‘.’ 0.1 ‘ ’ |
| | 1 |

The linear mixed model cannot be fitted. Alternatively, if the linear mixed model fails, then t-test for paired samples can be applied if the difference x-y follow normal distribution. Otherwise, Wilcoxon signed rank test can be applied.

| t.test(gent_wt_nm$entropy[ gent_wt_nm$group == “ctrl”], | |
|---|---|
| | gent_wt_nm$entropy[ gent_wt_nm$group == “nm”], |
| | paired = TRUE, |
| | alternative = “greater”) |
| #> | |
| #> | Paired t-test |
| #> | |
| #> | data: gent_{—}wt_{—}nm$entropy[gent_{—}wt_{—}nm$group == “ctrl”] and |
| | gent_{—}wt_{—}nm$entropy[gent_{—}wt |
| _{—}nm$group == “nm”] | |
| #> | t = − 2.2885, df = 24, p-value = 0.9844 |
| #> | alternative hypothesis: true difference in means is greater |
| | than 0 |
| #> | 95 percent confidence interval: |
| #> | −1.069629 Inf |
| #> | sample estimates: |
| #> | mean of the differences |
| #> | −0.6120537 |

Shapiro-Wilk normality test indicates that the Paired t-test result is not valid since the differences ‘d’ does not follow normal distribution.

| | |
|---|---|
| d = | gent_wt_nmsentropy[ gent_wt_nm$group == “ctrl” ] − |
| | gent_wt_nm$entropy[ gent_wt_nm$group == “nm” ] |

shapiro.test(d)
#>

| | |
|---|---|
| #> | Shapiro-Wilk normality test |

#>

| | |
|---|---|
| #> | data: d |
| #> | W = 0.75012, p-value = 3.727e−05 |

Alternatively, the Wilcoxon signed rank test, which does not depend on the normality hypothesis, can be applied.

wilcox.test(gent_wt_nm$entropy[ gent_wt_nm$group == “nm” ],
gent_wt_nmsentropy[ gent_wt_nm$group == “ctrl” ],
paired = TRUE,
alternative = “greater”)
#>

| | |
|---|---|
| #> | Wilcoxon signed rank exact test |

#>

| | |
|---|---|
| #> | data: gent_{—}wt_{—}nm$entropy[gent_{—}wt_{—}nm$group == “nm”] and |
| | gent_{—}wt_{—}nm$entropy[gent_{—}wt_{—}n m$group == “ctrl”] |
| #> | V = 266, p-value = 0.002088 |
| #> | alternative hypothesis: true location shift is greater than |
| | 0 |

### 3. Linear and Generalized Linear Models for Memory Line 3rd Generation

gent_wt_mm3_<− rbind (gofs$ctrl, gofs$mm3)
lm_wt_mm3_<− lmer (formula = entropy ~ group + (1|chr),
data = gent_wt_mm3)
summary(lm_wt_mm3)

| #> | Linear mixed model fit by REML. t-tests use Satterthwaite's | | | | | | |
|---|---|---|---|---|---|---|---|
| | method [ | | | | | | |
| #> | lmerModLmerTest] | | | | | | |
| #> | Formula: entropy ~ group +(1 \| chr) | | | | | | |
| #> | Data: gent_{—}wt_{—}mm3 | | | | | | |
| #> | | | | | | | |
| #> | REML criterion at convergence: 59.4 | | | | | | |
| #> | | | | | | | |
| #> | Scaled residuals: | | | | | | |
| #> | Min 1Q Median 3Q Max | | | | | | |
| #> | −1.9931 −0.4040 0.3778 0.7427 1.0794 | | | | | | |
| #> | | | | | | | |
| #> | Random effects: | | | | | | |
| #> | Groups | Name | Variance | Std. Dev. | | | |
| #> | chr | (Intercept) | 0.1666 | 0.4081 | | | |
| #> | Residual | | | | | | |
| #> | Number of obs: 50, groups: chr, 5 | | 0.1429 | 0.3780 | | | |
| #> | | | | | | | |
| #> | Fixed effects: | | | | | | |
| #> | | Estimate | Std. Error | | df | t value | Pr(>\|t\|) |
| #> | (Intercept) | −12.9888 | 0.1976 | | 4.6543 | −65.74 | 4.34e−08 *** |
| #> | groupmm3 | 2.6271 | 0.1069 | | 44.0000 ** | 24.57 | < 2e−16 * |
| #> | --- | | | | | | |
| #> | Signif. codes: 0 ‘***’ 0.001 ‘**’ 0.01 ‘*’ 0.05 ‘.’ 0.1 ‘ ’ | | | | | | |
| | 1 | | | | | | |
| #> | | | | | | | |

| | |
|---|---|
| #> | Correlation of Fixed Effects: |
| #> | (Intr) |
| #> | groupmm3 −0.271 |

anova(lm_{—}wt_{—}mm3)

| #> | Type III Analysis of Variance Table with Satterthwaite's | | | | | | |
|---|---|---|---|---|---|---|---|
| | method | | | | | | |
| #> | | Sum Sq | Mean Sq | NumDF | DenDF | F value | Pr(>F) |
| #> | group | 86.27 | 86.27 | 1 | 44 | 603.82 | < 2.2e−16 *** |
| #> | --- | | | | | | |

| | |
|---|---|
| #> | Signif. codes: 0 ‘***’ 0.001 ‘**’ 0.01 ‘*’ 0.05 ‘.’ 0.1 ‘ ’ |
| | 1 |

4. Linear and Nonlinear Models for met1

gent_wt_met1 <− rbind(gofs$ctrl_met1, gofs$met1)
glm_wt_met1 <− lmer(formula = entropy ~ group + (1|chr),
data = gent_wt_met1)

| #> | boundary(singular)fit: see ?isSingular | | | | | | | |
|---|---|---|---|---|---|---|---|---|
| | summary(glm_{—}wt_{—}met1) | | | | | | | |
| #> | Linear mixed model fit by REML. t-tests use Satterthwaite's | | | | | | | |
| | method [ | | | | | | | |
| #> | lmerModLmerTest] | | | | | | | |
| #> | Formula: entropy ~ group +(1 \| chr) | | | | | | | |
| #> | Data: gent_{—}wt_{—}met1 | | | | | | | |
| #> | | | | | | | | |
| #> | REML criterion at convergence: 84.7 | | | | | | | |
| #> | | | | | | | | |
| #> | Scaled residuals: | | | | | | | |
| #> | | Min | 1Q | Median | 3Q | Max | | |
| #> | | −1.0916 | −0.7395 | −0.4954 | 0.4192 | 2.2111 | | |
| #> | | | | | | | | |
| #> | Random effects: | | | | | | | |
| #> | Groups | | Name | Variance | | Std.Dev | | |
| #> | chr | | (Intercept) | 0.000 | | 0.0000 | | |
| #> | Residual | | | 0.642 | | 0.8013 | | |
| #> | Number of obs: 35, groups: chr, 5 | | | | | | | |
| #> | | | | | | | | |
| #> | Fixed effects: | | | | | | | |
| #> | | | Estimate | Std. Error | | df | t value | Pr(>\|t\|) |
| #> | (Intercept) | | −5.4721 | 0.1792 | | 33.0000 | −30.54 | <2e−16 *** |
| #> | groupmet1 | | 7.1851 | 0.2737 | | 33.0000 | 26.25 | <2e−16 *** |
| #> | --- | | | | | | | |
| #> | Signif. codes: 0 ‘***’ 0.001 ‘**’ 0.01 ‘*’ 0.05 ‘.’ 0.1 ‘ ’ | | | | | | | |
| | 1 | | | | | | | |
| #> | | | | | | | | |

| #> | Correlation of Fixed Effects: | |
|---|---|---|
| #> | | (Intr) |

| | |
|---|---|
| #> | groupmet1 −0.655 |
| #> | optimizer(nloptwrap)convergence code: 0(OK) |
| #> | boundary(singular)fit: see ?isSingular |

anova(glm_{—}wt_{—}met1)

| #> | Type III Analysis of Variance Table with Satterthwaite's | | | | | | |
|---|---|---|---|---|---|---|---|
| | method | | | | | | |
| #> | | Sum Sq | Mean Sq | NumDF | DenDF | F value | Pr(>F) |
| #> | group | 442.5 | 442.5 | 1 | 33 | 689.22 | < 2.2e−16 *** |
| #> | --- | | | | | | |

| | |
|---|---|
| #> | Signif. codes: 0 ‘***’ 0.001 ‘**’ 0.01 ‘*’ 0.05 ‘.’ 0.1 ‘ ’ |
| | 1 |

The linear mixed cannot be fitted. Alternatively, if the t-test paired samples can be applied if the difference x-y follow normal distribution. Since the there are more samples in the control than in ‘met1’ group, the first three samples from each chromosome from control group are taken for the paired comparison.

| wt_<− gent_wt_met1[ gent_wt_met1$group == “ctrl_met1”,] | | |
|---|---|---|
| idx <− is.element(wt$sample, c(“wt_1”, “wt_2”, “wt_3”)) | | |
| d = gent_wt_met1$entropy[ gent_wt_met1$group == “met1”] − | | |
| | wt$[ idx] | |
| shapiro.test(d) | | |
| #> | | |
| #> | Shapiro-Wilk normality test | |
| #> | | |
| #> | data: d | |
| #> | W = 0.9175, p-value = 0.1765 | |
| t.test(gent_wt_met1$entropy[ gent_wt_met1$group == “met1”], | | |
| | wt$entropy[ idx ], | |
| | paired = TRUE, | |
| | alternative = “greater”) | |
| #> | | |
| #> | Paired t-test | |
| #> | | |
| #> | data: gent_{—}wt_{—}met1$entropy[gent_{—}wt_{—}met1$group == “met1”] | |
| | and wt$entropy[idx] | |
| #> | t = 31.479, df = 14, p-value = 1. 073e−14 | |
| #> | alternative hypothesis: true difference in means is greater | |
| | than 0 | |
| #> | 95 percent confidence interval: | |
| #> | 6.591631 | Inf |
| #> | sample estimates: | |
| #> | mean of the differences | |
| #> | | 6.982298 |

5. Linear Model with Fixed Effects for met1

The linear model with fixed effects suggests that the failing of the linear mixed model would be originated by the fact that chromosomes effects (when considered as a fixed effects) is not statistically significantly.

gent_wt_met1 <− rbind(gofs$ctrl_met1, gofs$met1)
lm_wt_met1 <− lm(formula = entropy ~ group + chr,

data = gent_wt_met1)

summary(lm_wt_met1)

| #> | | | | | | |
|---|---|---|---|---|---|---|
| #> | Call: | | | | | |
| #> | lm(formula = entropy ~ group + chr, data = gent_{—}wt_{—}met1) | | | | | |
| #> | Residuals: | | | | | |
| #> | Min | 1Q | Median | 3Q | Max | |
| #> | −0.7507 | −0.5853 | −0.4474 | 0.3320 | 1.7349 | |
| #> | | | | | | |
| #> | Coefficients: | | | | | |
| #> | | | Estimate | Std. Error | t value | Pr(>\|t\|) |
| #> | (Intercept) | | −5.37591 | 0.34399 | −15.628 | 1.16e−15 *** |
| #> | groupmet1 | | 7.18508 | 0.28988 | 24.786 | < 2e−16 *** |
| #> | chrchr2 | | −0.22014 | 0.45364 | −0.485 | 0.631 |
| #> | chrchr3 | | −0.17607 | 0.45364 | −0.388 | 0.701 |
| #> | chrchr4 | | −0.09707 | 0.45364 | −0.214 | 0.832 |
| #> | chrchr5 | | 0.01251 | 0.45364 | 0.028 | 0.978 |
| #> | --- | | | | | |
| #> | Signif. codes: 0 ‘***’ 0.001 ‘**’ 0.01 ‘*’ 0.05 ‘.’ 0.1 ‘ ’ | | | | | |
| | 1 | | | | | |
| #> | | | | | | |

| #> | Residual standard error: 0.8487 on 29 degrees of freedom |
|---|---|
| #> | Multiple R-squared: 0.955, Adjusted R-squared: 0.9472 |

| | |
|---|---|
| #> | F-statistic: 123 on 5 and 29 DF, p-value: < 2.2e−16 |

anova(lm_{—}wt_{—}met1)

| #> | Analysis of Variance Table | | | | | |
|---|---|---|---|---|---|---|
| #> | | | | | | |
| #> | Response: entropy | | | | | |
| #> | | Df | Sum Sq | Mean Sq | F value | Pr(>F) |
| #> | group | 1 | 442.50 | 442.50 | 614.364 | <2e−16 *** |
| #> | chr | 4 | 0.30 | 0.07 | 0.104 | 0.9802 |
| #> | Residuals | 29 | 20.89 | 0.72 | | |
| #> | --- | | | | | |

#> Signif. codes: 0 '***' 0.001 1 '**' 0.01 '*' 0.05 '.' 0.1 ‘ ’

### 6. Result Table

gent <− function(s) {

| | t(sapply(s, gibb_entropy)) |
|---|---|
| } | |

## ===== Download methylation data from GitLab ========
samples <− c(“memory-col0-control−gen3”,

“non-memory-gen1”,
“memory-gen1”,
“memory-gen3”,
“col0-control-met1”,
“met1”)

url <− paste0(“https://git.psu. edu/genomath/datasets/-

/raw/main/at_mutants/”,“gofs/gof-jd-by-chr_”,
samples,“_all-contexts.RData”)

nms <− c(“ctrl”, “nm”, “mm1”, “mm3”, “msh1”, “ctrl_met1”,

“met1”)

entropy <− c( )
for (k in seq(samples)) {

| | temp <− tempfile(fileext = “.RData”) |
|---|---|
| | download.file(url = url[k], destfile = temp) |
| | load(temp) |
| | file.remove(temp) |
| | entropy <− rbind(entropy, gent(gof_jd)) |
| } | |

rm(temp, url)
colnames(entropy) <− paste0(“Chromosome ”, 1:5)
entropy

| #> | Chromosome 1 Chromosome 2 Chromosome 3 Chromosome 4 | | | | | |
|---|---|---|---|---|---|---|
| | Chromosome 5 | | | | | |
| #> | wt3_{—}1 | −12.094846 | −13.091612 | −12.853697 | −12.874774 | −12.397823 |
| #> | wt3_{—}2 | −12.238638 | −13.201601 | −12.826977 | −12.954719 | −12.446643 |
| #> | wt3_{—}3 | −12.581987 | −13.611254 | −13.311590 | −13.403394 | −12.871608 |
| #> | wt3_{—}4 | −12.190052 | −13.289105 | −12.883656 | −13.007626 | −12.534174 |
| #> | wt3_{—}5 | −13.009502 | −14.074439 | −13.805811 | −13.831105 | −13.333324 |
| #> | nm1_{—}1 | −10.516693 | −11.671416 | −11.429877 | −11.446873 | −10.970227 |
| #> | nm1_{—}2 | −10.344103 | −11.460553 | −11.193130 | −11.205235 | −10.757582 |
| #> | nm1_{—}3 | −13.423766 | −14.233640 | −14.126294 | −14.175181 | −13.761249 |
| #> | nm1_{—}4 | −10.331681 | −11.428398 | −11.160442 | −11.192328 | −10.739979 |
| #> | nm1_{—}5 | −14. 457926 | −14.972428 | −15.001781 | −14.803605 | −14.614226 |
| #> | m1_{—}1 | −12.452386 | −13.384541 | −13.152839 | −13.134270 | −12.807018 |
| #> | m1_{—}2 | −13.169982 | −14.111095 | −13.933727 | −13.978335 | −13.578820 |
| #> | m1_{—}3 | −10.484701 | −11.578029 | −11.390940 | −11.368858 | −10.946872 |
| #> | m1_{—}4 | −10.087015 | −11.177138 | −10.971958 | −10.981584 | −10.484795 |
| #> | m1_{—}5 | −9.969216 | −11.103592 | −10.818339 | −10.851584 | −10.297509 |
| #> | m3_{—}1 | −9.504246 | −10.593100 | −10.365805 | −10.370156 | −9.850248 |
| #> | m3_{—}2 | −9.617158 | −10.690615 | −10.536732 | −10.527998 | −10.013634 |
| #> | m3_{—}3 | −9.391835 | −10.474706 | −10.269382 | −10.263898 | −9.839075 |
| #> | m3_{—}4 | −10.335803 | −11.406912 | −11.292288 | −11.310295 | −10.824549 |
| #> | m3_{—}5 | −9.687667 | −10.736263 | −10.530887 | −10.526185 | −10.083295 |
| #> | wt_{—}1 | −3.751485 | −4.060561 | −3.957873 | −3.738039 | −3.700360 |
| #> | wt_{—}2 | −5.876468 | −6.242450 | −6.163961 | −5.959253 | −5.810828 |
| #> | wt_{—}3 | −5.869031 | −6.215879 | −6.070257 | −5.895751 | −5.727038 |
| #> | wt_{—}4 | −5.993948 | −6.346747 | −6.177509 | −5.994613 | −5.889276 |
| #> | met1_{—}1 | 2.183079 | 2.128942 | 2.065479 | 1.980010 | 2.085467 |
| #> | met1_{—}2 | 1.199392 | 1.126149 | 1.072355 | 1.004305 | 1.107616 |
| #> | met1_{—}3 | 2.032322 | 1.993428 | 1.923155 | 1.847710 | 1.945829 |

From the foregoing, it can be seen that the present disclosure accomplishes at least all of the stated objectives.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 6
List of Reference Characters

| | |
|---|---|
| ε | minimum energy dissipation |
| k_{B} | Boltzmann constant |
| T | absolute temperature |
| E | energy |
| Eᵢ | energy value |
| πᵢ | discrete probability to observe dissipation of the energy value |
| [symbol] E [symbol] | mathematical expectation of E |
| i | states |
| A | density |
| P | probability |
| f(E\|β, . . . ) | probability density function |
| β | scaling constant |
| C | machine capacity |
| P_{y} | energy dissipated by the molecular machine |
| N_{y} | energy of the thermal noise |
| d_{space} | number of independently moving parts of a molecular machine |
| E_{y} | energy average |
| Nᵢ | number of times that an amount of energy in an interval |
| k | number of events |
| pᵢ | probability that an amount of energy is dissipated in an interval |
| [E₁, E₂) | interval |
| [E_{k - 1}, Eₖ) | interval |
| α | parameter that carries information about the molecular machine |
| δ | molecular machine parameter |
| χ | information divergence |
| p | methylation level |
| q | methylation level |
| x | original message |
| y | receiving point |
| R | rate of generating information for a given quality |
| ρ(x, y) | distance function |
| ν | mean |
| σ | variance |
| nC^{m} | number of times that the cytosine is observed methylated at site |
| nCⁱ | number of times that the cytosine is observed unmethylated at site |
| ψ | digamma function |
| ϕ | function of a model parameter associated to the number of independent |
| | moving parts of the molecular machine |
| η | model parameter |
| μ | location parameter |
| S | Gibbs entropy |
| H | Shannon entropy |
| Iₘ | methylome gains information |
| G | Gibbs free energy |
| N | number of molecules |
| V | volume |
| T | temperature |
| F | Helmholtz free energy |
| S_{G} | Weibull distribution |
| F(β) | Gamma distribution |
| K_{B}⁻¹S | dimensionless entropy parameter |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

In biological systems, “methylation” is catalyzed by enzymes; such methylation can be involved in modification of heavy metals, regulation of gene expression, regulation of protein function, and RNA processing. In vitro methylation of tissue samples is also one method for reducing certain histological staining artifacts. The reverse of methylation is demethylation.

“DNA methylation” is a biological process by which methyl groups are added to the DNA molecule. Methylation can change the activity of a DNA segment without changing the sequence. When located in a gene promoter, DNA methylation can act to repress gene transcription. In mammals, DNA methylation is essential for normal development and is associated with a number of key processes including genomic imprinting, X-chromosome inactivation, repression of transposable elements, aging, and carcinogenesis.

A “methylome” is a set of nucleic acid methylation modifications in an organism's genome or in a particular cell.

“Epigenetics” is epigenetics the study of heritable phenotype changes that do not involve alterations in the DNA sequence. Epigenetics most often involves changes that affect gene activity and expression, but the term can also be used to describe any heritable phenotypic change. Such effects on cellular and physiological phenotypic traits may result from external or environmental factors, or be part of normal development. “Epigenetics” also refers to the changes themselves: functionally relevant changes to the genome that do not involve a change in the nucleotide sequence. Examples of mechanisms that produce such changes are DNA methylation and histone modification, each of which alters how genes are expressed without altering the underlying DNA sequence.

In information theory, the “entropy” of a random variable following a discrete probability distribution is the average level of “information”, “surprise”, or “amount of uncertainty” inherent to the variable's possible outcomes. Given a discrete random variable [symbol] , which takes values in the alphabet χ and is distributed according to and is distributed according to [symbol] →[0,1]:

$$H ( X ) = - {\sum }_{x ∈ 𝒳} p ( x ) log p ( x ) = 𝔼 [ - log p ( X ) ] ,$$

where Σ denotes the sum over the variable's possible values. The choice of base for log, the logarithm, varies for different applications. Base 2 gives the unit of bits; base e gives “natural units” nat; and base 10 gives units of “dits”. An equivalent definition of “entropy” is the expected value of the self-information of a variable.

For a random variable x with continuous probability distribution ƒ(x) the entropy is estimated as:

H(X)=∫₀^{∞}ƒ(x)ln ƒ(x)

The Gibb entropy S is then given as S=−k_{B}H(X), where k_{B} is the Boltzmann constant.

In information theory, the “Shannon-Hartley theorem” tells the maximum rate at which information can be transmitted over a communications channel of a specified bandwidth in the presence of noise. It is an application of the noisy-channel coding theorem to the archetypal case of a continuous-time analog communications channel subject to Gaussian noise. The theorem establishes Shannon's channel capacity for such a communication link, a bound on the maximum amount of error-free information per time unit that can be transmitted with a specified bandwidth in the presence of the noise interference, assuming that the signal power is bounded, and that the Gaussian noise process is characterized by a known power or power spectral density.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A methylation system utilizing methylation machinery to interpret messages in a framework of a communication system comprising:
a methylome dataset relating to a molecular machine;
a generalized gamma distribution model to describe genome-wide methylation changes observable in the methylome dataset, said generalized gamma distribution model comprising a probability density function (ƒ(E|β, . . . )) and expressed in terms of an information divergence (χ) of methylation changes, wherein the information divergence (χ) (1) is proportional to an energy (Eᵢ) dissipated per bit of information in the methylation changes and (2) holds a symmetry axiom;
an instrument for calculating or measuring entropy (S) derived from a state (i) of the methylation system, said entropy (S) being based on the information divergence (χ).

2. The methylation system of claim 1, wherein the molecular machine:
is an enzyme;
includes a capacity (C) that is related to a maximum amount of information that a molecular machine gains per operation; or
includes independently moving parts that are involved in the operation, wherein a number of said independently moving parts is proportional to the capacity (C) of the molecular machine.

3. The methylation system of claim 2, wherein:
a minimum energy dissipation per bit of information per machine operation, at a normal temperature of the human body, is at least 1.784 J·mol⁻¹;
the probability to observe a methylation change will decline with the increment of an amount of energy dissipated per bit of information processed by the molecular machines, wherein the entropy(S) of the methylation system is output as a classical term (S_{classic}) and a contribution from the independently moving parts (S_{machine}); or
the capacity (C) can be increased by increasing an energy (E_{y}) relative to an energy of the thermal noise (N_{y}).

4. The methylation system of claim 1, further comprising a quantifier that summarizes statistical physics underlying the methylation changes that are not induced by the methylation machinery.

5. The methylation system of claim 1, further comprising:
an application of thermodynamic principles to chromatin dynamics on a DNA molecule maximizes Boltzmann entropy, leading, in turn, to an identification of most probable methylation density states for the methylation system;
wherein:
the most probable methylation density states for the methylation system is determined by a number of cytosine sites in the DNA molecule;
the methylation system is constrained by a discrete probability (πᵢ) to observe dissipation of an energy value (Eᵢ);
the methylation system is constrained by a mathematical expectation ( [symbol] E [symbol] ) of energy (E);
the discrete probability (πᵢ) to observe a genome-wide energy dissipation between 0 and the energy (E) is inversely proportional to a partition function of the methylation system that includes a temperature (T) dependent scaling constant (β);
probabilities (pᵢ) that an amount of energy (E) is dissipated in an interval ([E₁, E₂), . . . , [Eₖ₋₁, Eₖ)) are proportional to the energy value (Eᵢ); or
for each choice of a parameter (α) that carries information about the molecular machine and effects the energy value (Eᵢ), a sum of a number of times (Nᵢ) that the energy (E) energy in the interval ([E₁, E₂), . . . , [Eₖ₋₁, Eₖ)) and the energy value (Eᵢ^{α}) are positive.

6. The methylation system of claim 1, further comprising an output that expresses the information divergence (χ) of methylation changes selected from the group consisting of:
Hellinger divergence;
J-divergence;
total variation divergence (TV);
total variation distance (TVD); and
cross entropy.

7. The methylation system of claim 1, further comprising code describing a genome-wide patterning of DNA methylation that occurs at specific landmarks.

8. The methylation system of claim 1, wherein:
the code describes a conditional probability (P_{xy}), so that if a message (x) is produced by a source, the message can be recovered at a receiving point (y); and
the message (x) transmitted is expressed at each cytosine site in terms of observed methylation levels (x, y) in a treatment or a patient group.

9. The methylation system of claim 8, wherein:
the methylation levels (x, y) are estimated as a percentage of the number of times that the cytosine is observed methylated (nC^{m}) and unmethylated (nCᵢ) at a site (i); and
the information divergence (χ) is a symmetric information divergence (χ(x, y)) about the methylation levels (x and y).

10. The methylation system of claim 1, wherein the entropy (S) is a rough estimate for different tissues/cells and is based on the information divergence (χᵢ) after expressing the energy value (Eᵢ) in terms of the information divergence (χᵢ) according to the probability density function (ƒ(E|β, . . . )).

11. The methylation system of claim 1, wherein:
the entropy (S) is proportional to a Shannon entropy (H) at a constant temperature (T);
the Shannon entropy (H) depends only on distribution parameters estimated from individualistic-based data within the methylome dataset; or
the instrument for calculating or measuring entropy (S) can quantify methylome gains information (Iₘ) and can determine whether there has been a loss or a gain of information.

12. The methylation system of claim 11, further comprising:
(a) pseudorandom numbers, (b) a Monte Carlo simulation and resampling, or (c) a bootstrap or numerical approach that allows the computation of the entropy (S) (1) without the estimation of GGamma parameters via numerical integration algorithms or (2) with an acceptable estimation of Gibbs or Helmholtz free energy (G, F)
wherein the instrument for calculating or measuring entropy (S) uses an empirical cumulative distribution function (ECDF) and a kernel density estimation algorithm with a cubic spline function to estimate an empirical density function (EDF).

13. The methylation system of claim 1, further comprising:
an estimator of Helmholtz free energy (F) that utilizes the entropy (S) to further determine a thermodynamic potential of the closed methylation system;
wherein:
the methylome dataset is large enough such that a balance exists between methylation and demethylation processes along each DNA molecule, or
an overall mass determined by a number of the DNA molecules (N), a volume of the DNA molecules (V), and a temperature (T) of the DNA molecules are assumed to be constant, thereby making the methylation system a closed system.

14. The methylation system of claim 13, wherein the estimator is an R package that includes functions for entropy (S) and Helmholtz free energy (F) estimations, given by the rough estimate of Gibbs free energy (G) for different tissues/cells and the change in Helmholtz free energy (F).

15. The methylation system of claim 1, further comprising:
an estimator of Gibbs free energy (G) that utilizes Shannon entropy (H) to further determine a thermodynamic potential of the closed methylation system.

16. The methylation system of claim 1, further comprising a device to observe phenotypic changes that coincide with the entropy (S).

17. The methylation system of claim 1, wherein:
the methylome dataset relates to information about human genes; and
the methylome dataset includes information selected from the group consisting of:
information about naïve human pluripotent cells;
information about human cancer types and corresponding normal tissue selected form the group consisting of: brain white matter, brain Glioma, breast normal, breast cancer, breast metastasis, colon normal, colon cancer, colon metastasis, lung normal, lung cancer, normal blood B-cells, and placenta.

18. The methylation system of claim 1, wherein:
the methylome dataset relates to plant genes; and
the methylome dataset includes information about a transgene null plant experiencing a change in a phenotype selected from the group consisting of:
a. a time for flowering;
b. an overall size of the transgene plant; and
c. a color of the plant.

19. A method of utilizing thermodynamics of a DNA methylation process comprising:
discriminating a methylation regulatory signal from background noise; and
assessing thermodynamics of a methylation variation in a population of cells by observing:
statistical physics underlying methylation changes; and
a methylation regulatory machinery.

20. An extension for a general-purpose programming language or a statistical programming language, said extension comprising:
algorithm(s) that analyze thermodynamics of methylation signals on stretches of DNA sequences, said DNA sequences being characterized by:
i. methylation information; and
ii. physicochemical information around each methylated cytosine;
wherein the algorithm(s) include one or more functions that can:
estimate a probability to observe a genome-wide energy dissipation between 0 and energies E;
estimate a probability to observe a genome-wide information divergence between 0 and χ;
use a probability density function of DNA methylation information-divergence to quantitatively summarize statistical physics underlying methylation changes;
calculate a conditional probability that a recovered message at a receiving point is produced by the source;
determine a thermodynamic potential of a closed system at a constant temperature and volume.
